(19) United States
(12) Patent Application Publication
(10) Pub. No.: US 2025/0329431 A1
(43) Pub. Date: Oct. 23, 2025

(54) METHOD OF PREDICTING NON-SMALL CELL LUNG CANCER (NSCLC) PATIENT DRUGRESPONSE OR TIME UNTIL DEATH OR CANCER PROGRESSION FROM CIRCULATING TUMORDNA (CTDNA) UTILIZING SIGNALS FROM BOTH BASELINE CTDNA LEVEL AND LONGITUDINALCHANGE OF CTDNA LEVEL OVER TIME

(71) Applicant: GUARDANT HEALTH, INC., Palo Alto, CA (US)

(72) Inventors: Bojan LOSIC, Hartford, CT (US); Katie Julia QUINN, Redwood City, CA (US); Sean Gordon, San Diego, CA (US)

(21) Appl. No.: 19/205,670

(22) Filed: May 12, 2025

Related U.S. Application Data
(63) Continuation of application No. PCT/US2023/079340, filed on Nov. 10, 2023.
(60) Provisional application No. 63/383,838, filed on Nov. 15, 2022.
(60) Provisional application No. 63/493,075, filed on Mar. 30, 2023.

(51) Int. Cl. G16H 20/17; G06N 20/00; G16B 20/20; G16B 40/20; G16H 50/70

(52) U.S. Cl. CPC G16H 20/17; G06N 20/00; G16B 20/20; G16B 40/20; G16H 50/70

(57) ABSTRACT

Provided herein are methods of determining a molecular response score for use in predictive models. The molecular response score may be used to monitor and guide administration of treatment to a subject.

### CROSS-REFERENCE TO RELATED APPLICATIONS

The present application is a Continuation of International Patent Application No. PCT/US2023/079340, filed Mar. 30, 2023, which claims the benefit of priority of U.S. Provisional Patent Application No. 63/383,838, filed Nov. 15, 2022, and, U.S. Provisional Patent Application No. 63/493,075, filed Mar. 30, 2023, which are incorporated by reference herein in its entirety for all purposes.

### BACKGROUND

Molecular response is a calculation of the change in circulating tumor DNA (ctDNA) levels observed in samples collected from subjects at different time points. In certain cases, the calculation is based on the fraction of somatic variants in the total cell-free DNA (cfDNA) in samples. In other cases, the calculation is based on the concentration of ctDNA in the samples (i.e., normalized per the cfDNA concentration in the samples). A common problem is that existing calculations of molecular response frequently yield inaccurate or imprecise molecular response scores. Additionally, there is little information on how these variables can be combined to better interpret ctDNA results and improve prediction of patient response to treatment or patient progression. Prediction of patient response to therapy or cancer progression is important information for clinicians who may use such results to alter treatment of the patient to more/less aggressive options depending on the result. Thus, there remains a need for methods for determining molecular response scores with meaningful interpretation of clinical significance such as correlation of changes in ctDNA quantity correlate with response to therapy and absolute baseline (pre-treatment) ctDNA level has been shown to be associated with cancer patient prognosis.

Described herein is a model that incorporates the effects of baseline ctDNA level and its interaction with linear and nonlinear relative ctDNA change to predict the time duration until a patient will experience a death or progression event given patient attributes and/or ctDNA measurements of baseline ctDNA level and a metric of ctDNA change.

### SUMMARY OF THE INVENTION

Described herein is a method, comprising receiving, by a computer system, genetic information of a subject comprising data taken at two or more time points and wherein cancer is detected in the subject, extracting one or more features from the genetic information, the one or more features identified from a plurality of samples obtained from the subject; generating, by a first classifier implemented by the computer system using a first machine learning algorithm, a first output indicating a first classification of the subject; generating, by a second classifier implemented by the computer system using a second machine learning algorithm, a second output indicating a second classification of the subject; identifying, by the computer system and from a population, additional subjects with genetic information that match the subject's genetic information based on the first classification and the second classification; determining, by the computer system, at least one score with respect to the subject based additional subjects with matching genetic information; determining, by the computer system, a composite score using the at least one score; and determining, by a recommender implemented by the computer system, a recommendation indicating a treatment for the subject based on the composite score.

In other embodiments, the one or more features comprise a first mutant allele fraction (MAF) and a second MAF, each from the at two or more time points. In other embodiments, the at least one score is based on a first mutant allele fraction (MAF) and a second MAF, a weighted mean of the first MAFs and a weighted mean of the second MAFs. In other embodiments, the least one score is based on the ratio of the weighted mean of the first MAFs and the weighted mean of the second MAFs and the confidence interval. In other embodiments, the at least one score is based on a first mutant allele fraction (MAF) at the first time point and a second MAF at the second time point, a first central tendency measure of the first MAFs and a second central tendency measure of the second MAFs; In other embodiments, the at least one score is based on the ratio of the first central tendency measure at the first time point to the second central tendency measure at the second time point. In other embodiments, the central tendency measure is one or more of a: mean, median, or mode. In other embodiments, the method includes comparing the molecular response score for the subject having the cancer to a predetermined cutoff point to identify that the subject is a likely responder to one or more therapies for the cancer when the molecular response score is below the predetermined cutoff point or that the subject is a likely non-responder to the one or more therapies for the cancer when the molecular response score is at or above the predetermined cutoff point. In other embodiments, the one or more therapies comprise one or more immunotherapies. In other embodiments, the method includes administering one or more therapies for the cancer to the subject in view of the at least one score. In other embodiments, the discontinuing administering one or more therapies for the cancer to the subject in view of the at least one score. In other embodiments, the method includes using the at least one score as a prognostic biomarker and/or a predictive biomarker for the subject. In other embodiments, the method includes using a molecule count to calculate the standard deviation for each MAF ratio in the set of MAF ratios. In other embodiments, the method includes propagating a variance through each MAF ratio in the set of MAF ratios.

In other embodiments, the method includes one or more germline and/or clonal hematopoietic variants when determining the mutant allele frequencies (MAFs). In other embodiments, the first time point comprises a pre-treatment time point and wherein the second time point comprises an on- or post-treatment time point. In other embodiments, the method includes generating the sequence information from nucleic acid molecules obtained from one or more tissues or cells in the sample. In other embodiments, the method includes generating the sequence information from cell-free nucleic acids (cfNAs) in the samples obtained from the subject. In other embodiments, the cfNAs comprise circulating tumor DNA (ctDNA). In other embodiments, the first and/or second classifier is each implemented by a machine learning algorithm. In other embodiments, the machine learning algorithm is selected from a neural network, a support vector machine, a Hidden Markov Model, or a random forest model. In other embodiments, the at least one score corresponds to a level of responsiveness to a treatment from a plurality of levels of responsiveness to the treatment.

Described herein is a method, comprising: using a genetic analyzer to generate genetic information; receiving, into computer memory, a training dataset comprising, for each of a plurality of individuals having a cancer disease: (1) genetic information from the individual generated at first time point and (2) treatment response of the individual to one or more therapeutic interventions determined at a second, later, time point; using the training dataset to subject a computer classifier to training to yield a trained computer classifier, wherein the trained computer classifier is configured to: determine at least one score of a SUBJECT based on at least mutant allele frequencies (MAFs) at the first time point and second time point; determine an amount of change between MAFs; In other embodiments, the method includes predicting a therapeutic response for the subject based on the amount of change between MAFs.

In other embodiments, the method includes selecting a treatment for the subject based on the amount of change between MAFs.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads and the second plurality of sequence reads as somatic or germline, determining, for at least one variant of the plurality of variants classified as somatic, based on a first mutant allele fraction (MAF) and a second MAF, a weighted mean of the first MAFs and a weighted mean of the second MAFs, determining, for the subject, a ratio of the weighted mean of the first MAFs and the weighted mean of the second MAFs, determining, based on the ratio of the weighted mean of the first MAFs and the weighted mean of the second MAFs, a confidence interval, and outputting, as a molecular response score, the ratio of the weighted mean of the first MAFs and the weighted mean of the second MAFs and the confidence interval.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads and the second plurality of sequence reads as somatic or germline, determining, for at least one variant of the plurality of variants classified as somatic, based on a first mutant allele fraction (MAF) and a second MAF, an MAF ratio, determining, for the subject, a weighted mean of the MAF ratios, determining, based on the weighted mean of the MAF ratios, a confidence interval associated with the weighted mean of the MAF ratios, and outputting, as a molecular response score, the weighted mean of the MAF ratios and the confidence interval.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads as somatic or germline, classifying the plurality of variants in the second plurality of sequence reads as somatic or germline, reclassifying at least one variant of the plurality of variants to resolve a classification discrepancy between the first plurality of sequence reads and the second plurality of sequence reads, determining, for at least one variant of the plurality of variants classified or reclassified as somatic, based on at least a portion of the first plurality of sequence reads, a first mutant allele fraction, determining, for at least one variant of the plurality of variants classified or reclassified as somatic, based on at least a portion of the second plurality of sequence reads, a second mutant allele fraction, and determining, based on the first mutant allele fraction and the second mutant allele fraction, a molecular response score.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads and the second plurality of sequence reads as somatic or germline, determining at least one variant of the plurality of variants as a Clonal Hematopoiesis of Indeterminate Potential (CHIP) variant, removing, from the plurality of variants, the at least one CHIP variant, determining, for at least one variant of the plurality of variants classified as somatic, based on at least a portion of the first plurality of sequence reads, a first mutant allele fraction, determining, for at least one variant of the plurality of variants classified as somatic, based on at least a portion of the second plurality of sequence reads, a second mutant allele fraction, and determining, based on the first mutant allele fraction and the second mutant allele fraction, a molecular response score.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads as somatic or germline, classifying the plurality of variants in the second plurality of sequence reads as somatic or germline, reclassifying at least one variant of the plurality of variants to resolve a classification discrepancy between the first plurality of sequence reads and the second plurality of sequence reads, determining at least one variant of the plurality of variants as a Clonal Hematopoiesis of Indeterminate Potential (CHIP) variant, removing, from the plurality of variants, the at least one CHIP variant, determining, for at least one variant of the plurality of variants classified or reclassified as somatic, based on at least a portion of the first plurality of sequence reads, a first mutant allele fraction, determining, for at least one variant of the plurality of variants classified or reclassified as somatic, based on at least a portion of the second plurality of sequence reads, a second mutant allele fraction, determining, for at least one variant of the plurality of variants classified or reclassified as somatic, based on the first mutant allele fraction and the second mutant allele fraction, an MAF ratio, determining, for the subject, a weighted mean of the MAF ratios, determining, based on the weighted mean of the MAF ratios, a confidence interval associated with the weighted mean of the MAF ratios, and outputting, as a molecular response score, the weighted mean of the MAF ratios and the confidence interval.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads as somatic or germline, classifying the plurality of variants in the second plurality of sequence reads as somatic or germline, reclassifying at least one variant of the plurality of variants to resolve a classification discrepancy between the first plurality of sequence reads and the second plurality of sequence reads, determining at least one variant of the plurality of variants as a Clonal Hematopoiesis of Indeterminate Potential (CHIP) variant, removing, from the plurality of variants, the at least one CHIP variant, determining, for at least one variant of the plurality of variants classified as somatic, based on at least a portion of the first plurality of sequence reads, a first mutant allele fraction (MAF), determining, for at least one variant of the plurality of variants classified as somatic, based on at least a portion of the second plurality of sequence reads, a second MAF, determining, for the at least one variant of the plurality of variants classified as somatic, based on the first MAF and the second MAF, a weighted mean of the first MAFs and a weighted mean of the second MAFs, determining, for the subject, a ratio of the weighted mean of the first MAFs and the weighted mean of the second MAFs, determining, based on the ratio of the weighted mean of the first MAFs and the weighted mean of the second MAFs, a confidence interval, and outputting, as a molecular response score, the ratio of the weighted mean of the first MAFs and the weighted mean of the second MAFs and the confidence interval.

In an aspect, this disclosure provides a method of determining a molecular response score at least partially using a computer. The method includes determining a first plurality of sequence reads and a second plurality of sequence reads associated with a subject, wherein the first plurality of sequence reads are determined at a first time point before administering a therapy and the second plurality of sequence reads are determined at a second time point after administering the therapy, classifying a plurality of variants in the first plurality of sequence reads and the second plurality of sequence reads as somatic or germline, determining, for at least one variant of the plurality of variants classified as somatic, based on a first mutant allele fraction (MAF) at the first time point and a second MAF at the second time point, a first central tendency measure of the first MAFs and a second central tendency measure of the second MAFs, determining a ratio of the first central tendency measure at the first time point to the second central tendency measure at the second time point, and outputting, as a molecular response score, the ratio of the first central tendency measure at the first time point to the second central tendency measure at the second time point.

In one aspect, this disclosure provides a method of determining a molecular response score for a subject having cancer at least partially using a computer. The method includes (a) determining, by the computer, mutant allele frequencies (MAFs) for a plurality of variants from sequence information generated from targeted nucleic acids associated with one or more cancer types in samples obtained from the subject at first and second time points to produce sets of first and second MAFs for each variant in the plurality of variants. The method also includes (b) calculating, by the computer, a ratio of the first and second MAFs for each variant in the plurality of variants to produce a set of MAF ratios and a corresponding standard deviation for each MAF ratio in the set of MAF ratios. In addition, the method also includes (c) calculating, by the computer, a weighted mean of the MAF ratios and a confidence interval, thereby determining the molecular response score for the subject having the cancer.

In another aspect, this disclosure provides a method of treating cancer in a subject. The method includes (a) determining mutant allele frequencies (MAFs) for a plurality of variants from sequence information generated from targeted nucleic acids associated with one or more cancer types in samples obtained from the subject at first and second time points to produce sets of first and second MAFs for each variant in the plurality of variants. The method also includes (b) calculating a ratio of the first and second MAFs for each variant in the plurality of variants to produce a set of MAF ratios and a corresponding standard deviation for each MAF ratio in the set of MAF ratios. The method also includes (c) calculating a weighted mean of the MAF ratios and a confidence interval to determine a molecular response score for the subject. In addition, the method also includes (d) administering one or more therapies to the subject based upon at least the molecular response score, thereby treating the cancer in the subject.

In another aspect, this disclosure provides a method of treating cancer in a subject. The method includes administering one or more therapies to the subject based upon at least a molecular response score for the subject. The molecular response score is produced by: (a) determining, by a computer, mutant allele frequencies (MAFs) for a plurality of variants from sequence information generated from targeted nucleic acids associated with one or more cancer types in samples obtained from the subject at first and second time points to produce sets of first and second MAFs for each variant in the plurality of variants; (b) calculating, by the computer, a ratio of the first and second MAFs for each variant in the plurality of variants to produce a set of MAF ratios and a corresponding standard deviation for each MAF ratio in the set of MAF ratios; and (c) calculating, by the computer, a weighted mean of the MAF ratios and a confidence interval to determine the molecular response score for the subject.

In another aspect, this disclosure provides a method of identifying clonal hematopoietic variants in a subject having cancer at least partially using a computer. The method includes (a) determining, by the computer, a tumor load change (R) for tumor fraction change P(R) for each of a plurality of variants from sequence information generated from targeted nucleic acids associated with one or more cancer types in samples obtained from the subject at first and second time points to produce a set of tumor load changes. The method also includes (b) identifying, by the computer, one or more resistance signatures corresponding to one or more clonal hematopoietic variants from the set of tumor load changes, thereby identifying the identifying the clonal hematopoietic variants in the subject having cancer.

In another aspect, this disclosure provides a method of identifying clonal hematopoietic variants in a subject having cancer at least partially using a computer. The method includes (a) calculating, by the computer, a probability density function for tumor fraction change P(R) for each of a plurality of variants from sequence information generated from targeted nucleic acids associated with one or more cancer types in samples obtained from the subject at first and second time points. The method also includes (b) grouping, by the computer, one or more of the variants by P(R) into one or more clones, and (c) generating, by the computer, an updated P(R) for each of the clones. In addition, the method also includes (d) identifying, by the computer, one or more clones having a fractional change between the first and second time points at or above a predetermined threshold value, thereby identifying the identifying the clonal hematopoietic variants in the subject having cancer. In some of these embodiments, the method includes determining a likelihood that a given pair of variants exhibit an identical fractional change, merging most likely pairs of variants into one clone, and updating the P(R) for the one clone.

In another aspect, this disclosure provides a method of identifying germline variants in a subject having cancer at least partially using a computer. The method includes (a) determining, by the computer, a mutant allele frequency (MAF) for a given variant from sequence information generated from targeted nucleic acids associated with one or more cancer types in a sample obtained from the subject. The method also includes (b) identifying, by the computer, that the given variant is a germline variant when the MAF of the given variant increases the max MAF of the sample, which sample comprises a max fraction of diploid genes (max frac_diploid) and/or when the MAF of the given variant is at least about two times greater, three times greater, four times greater, five times greater, six times greater, seven times greater, eight times greater, nine times greater, or more than one or more other MAFs determined from the sample obtained from the subject, thereby identifying the germline variants in the subject having cancer.

In some embodiments, the methods disclosed herein include comparing the molecular response score for the subject having the cancer to a predetermined cutoff point to identify that the subject is a likely responder to one or more therapies for the cancer when the molecular response score is below the predetermined cutoff point or that the subject is a likely non-responder to the one or more therapies for the cancer when the molecular response score is at or above the predetermined cutoff point. In some embodiments, the one or more therapies comprise one or more immunotherapies. In some embodiments, the methods disclosed herein include administering one or more therapies for the cancer to the subject in view of the molecular response score. In some embodiments, the methods disclosed herein include discontinuing administering one or more therapies for the cancer to the subject in view of the molecular response score. In some embodiments, the methods disclosed herein include recommending one or more therapies. In some embodiments, the methods disclosed herein include recommending discontinuing one or more therapies. In some embodiments, the methods disclosed herein include using the molecular response score as a prognostic biomarker and/or a predictive biomarker for the subject.

In some embodiments, the methods disclosed herein include using a molecule count to calculate the standard deviation for each MAF ratio in the set of MAF ratios. In some embodiments, the methods disclosed herein include propagating a variance through each MAF ratio in the set of MAF ratios. In some embodiments, the methods disclosed herein include excluding one or more germline and/or clonal hematopoietic variants when determining the mutant allele frequencies (MAFs) for the plurality of variants. In some embodiments, the plurality of variants comprises somatic nucleic acid variants. In some embodiments, the methods disclosed herein include excluding one or more somatic variants having MAFs that are less than about 0.1%, 0.2%, 0.3%, 0.4%, 0.5%, 0.6%, 0.7%, 0.8%, or 0.9% at both the first and second time points. In some embodiments, the first time point comprises a pre-treatment time point and wherein the second time point comprises an on- or post-treatment time point.

In some embodiments, the methods disclosed herein include generating the sequence information from nucleic acid molecules obtained from one or more tissues or cells in the sample. In some embodiments, the methods disclosed herein include generating the sequence information from cell-free nucleic acids (cfNAs) in the samples obtained from the subject. In some embodiments, the cfNAs comprise circulating tumor DNA (ctDNA).

In some embodiments, the ratio comprises the second MAF to the first MAF for each variant in the plurality of variants. In some embodiments, the methods disclosed herein include calculating the weighted mean of the MAF ratios using the formula: sum [weight*ratio]/sum [weights], where weight is 1/range2 for a given variant in the plurality of variants, where range is a difference between values of the first and second MAFs for a given variant in the plurality of variants, and ratio is a given MAF ratio in the set of MAF ratios. In some embodiments, the methods disclosed herein include calculating the confidence interval using the formula: weighted mean of the MAF ratios+/−sqrt [ratio variance], where ratio variance is 1/sum [weights].

In some embodiments, the variants comprise one or more single-nucleotide variants (SNV), insertion/deletion mutations (indels), gene amplifications, and/or gene fusions. In some embodiments, the methods disclosed herein include using one or more additional genomic data sources to determine the molecular response score for the subject having the cancer. In some embodiments, the additional genomic data sources comprise one or more of: a coverage, an off-target coverage, an epigenetic signature, and/or a microsatellite instability score. In some embodiments, the epigenetic signature comprises a cfNA fragment length, position, and/or endpoint density distribution. In some embodiments, the epigenetic signature comprises an epigenetic state or status exhibited by one or more epigenetic loci in a given targeted genomic region. In some embodiments, the epigenetic state or status comprises a presence or absence of methylation, hydroxymethylation, acctylation, ubiquitylation, phosphorylation, sumoylation, ribosylation, citrullination, and/or a histone post-translational modification or other histone variation.

This application discloses methods, computer readable media, and systems that are useful in determining molecular response scores for subjects having cancer. Related methods of identifying clonal hematopoietic and/or germline variants are also disclosed. Additional advantages of the disclosed method, systems, and/or compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1. Visualization of the CPH model (utilizing splines for MR) for predicting whether a patient will have a TTNT event. The model is specified to use interaction between ctDNA change (using Guardant molecular response score; MR Score=mean treatment VAF/mean baseline VAF) and ctDNA level variable (mean baseline VAF). Plot shows how ctDNA change interacts with baseline and ctDNA level to affect the response in the CPH regression model. Plot shows the relationship between the outcome (TTNT) and the explanatory variables (ctDNA change interacts with baseline and ctDNA level) while the other variables are held constant (weighted comorbidity score=21, female gender, patient age=66).

FIG. 2. Forest plot of hazard (HR) for death from CPH for molecular responders, defined at varying thresholds or ctDNA change (from 90 decrease at top to 20% decrease at the bottom). This figure is for dataset 2 ICI cohort and −60% (60% decrease in ctDNA) was chosen as the optimal genomic MR threshold to define MR responder/non-responder for the ICI treated patient cohort. All the respective thresholds below were significant for TTNT. Figure shows significantly reduced HR for OS when patients achieved 50% or more reduction in ctDNA. In contrast to OS, all above thresholds were significant for longer rwTTNT (reduction of 20% or more in ctDNA).

FIG. 3. Patients with “responder” and “non-responder” categories based on ctDNA change metric alone can be further sub divided by their baseline ctDNA level (high/low), resulting in 4 categories that better associate with both real world OS and TTNT. Non-responders with high levels of baseline ctDNA consistently have the shortest OS and TTNT in both the ICI and TKI treatment cohorts. A) Patients within ICI and TKI treatment cohorts were categorized into 4 categories: responder/low baseline (R/L), responder/high baseline (R/H), non-responder/low baseline (N/L), non-responder/high baseline (N/H) based on molecular responder (R)/non-responder (N) and high (H) or low (L) baseline ctDNA level. Patient counts and plots reflect thresholds optimized for rwTTNT individually. B-C) Maximum variant allele fraction (MVAF) is plotted (mean and 95% CI) between baseline and on-treatment timepoint for respective MR/MVAF groups within ICI cohort (B) and TKI cohort (C). Kaplan Meier plots of rwOS (D) and rwTTNT (F) in the ICI cohort and rwOS (E) and rwTTNT (G) in the TKI cohort.

FIG. 4. One year survival probability for rwOS and rwTTNT for groups in FIG. 3. Patients with ctDNA change metric alone can be further sub divided by their baseline ctDNA level (high/low). Patients with low baseline ctDNA and decreasing in ctDNA change metric have higher percentage of patients without OS and TTNT event within 1 year, whereas patients with high baseline ctDNA and increasing in ctDNA change metric have lowest percentage of patients without OS and TTNT event within 1 year. Molecular responders (R, white) and nonresponders (N, black), low baseline ctDNA (L, maroon), high baseline ctDNA (H, grey), responder/low baseline (R/L, orange), responder/high baseline (R/H, green), non-responder/low baseline (N/L, red), non-responder/high baseline (N/H, blue) in ICI (A,B) and TKI cohorts (C,D).

### DETAILED DESCRIPTION

The disclosed method and compositions may be understood more readily by reference to the following detailed description of particular embodiments and the Examples included therein and to the Figures and their previous and following description.

It is to be understood that the disclosed method and compositions are not limited to specific synthetic methods, specific analytical techniques, or to particular reagents unless otherwise specified, and, as such, may vary.

The present invention relates to compositions and methods for cancer diagnosis, research and therapy, including but not limited to, cancer markers. In particular, the present invention relates to predicting the likelihood of patient progression via computational modeling that incorporates both baseline ctDNA level as well as measures of ctDNA change in the same predictive model. Baseline ctDNA level can be interpreted broadly as a measure of ctDNA from a timepoint prior to the last timepoint within a series (2 or more) of measurements at different timepoints. The current work focuses on ctDNA measurements based on genomic alterations, but measures of ctDNA based on DNA methylation could be used in place of genomic alterations as long as they quantify the amount of ctDNA within a sample at a given timepoint.

Previous studies have attempted linear combinations of individual features and their association with patient outcomes such as PFS and OS. The current work differs from prior studies in the successful conception and implementation of predictive model that incorporates explicit interactions between baseline ctDNA level and a measure of ctDNA change between timepoints. The importance of ctDNA change within the predictive model can vary depending on the value of the baseline ctDNA level.

Trained models developed in this work may be used to predict patient outcome in terms of time to OS event and time to progression event (TTNT) for clinical patients in order for clinicians to determine what is the optimal treatment option for a given patient.

Patients at high risk for OS event or progression event from the model may be switched from current treatment to an alternative treatment which may be more aggressive treatment regimens e.g. ICI combined with chemotherapy rather than ICI mono therapy. Likewise patients at low risk of OS event or progression event may be switched less aggressive treatment options e.g. ICI mono therapy rather than ICI combined with chemotherapy.

Predictions from modeling the model described in this work may be translated into reports that quantify the risk of a cancer patient for death or progression on treatment. There are many different embodiments in how risk can be quantified and communicated from the predictive model. Categorical risk levels may be derived from the predictive model by use of thresholds on the amount of time predicted until a OS or progression (TTNT) event. For example, patients that are predicted to have an OS or TTNT event within the shortest quartile may be categorized as “high risk”, whereas patients with the longest quartile of predicted time until OS or TTNT event may be categorized as “low risk”. The clinician is then able to immediately utilize the information in order to optimize the care of the subject. Alternatively the predicted time until OS or TTNT may be provided as a integer score in which higher score is associated with lower risk, for example.

### I. Terms and Abbreviations

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).
CI=confidence interval
CPH=Cox proportional hazards model
ctDNA=circulating cell-free tumor DNA
G360=Guardant360, HR=hazard ratio
ICI=immune checkpoint inhibitor
TKI=Tyrosine Kinase inhibitors
rwTTNT=real world time to next treatment. Time from treatment start until the start of the next treatment, death, or lost to follow up.
Rw TTD=time to treatment discontinuation. Time from treatment start until the end of that treatment regimen, death, or lost to follow up.
LOT=line of therapy
AIC=Akaike Information Criterion.

Also it is understood by one of skill that terms used interchangeably including “maximum mutant allele frequency,” “maximum variant allele frequency,” “maximum MAF,” “MAX MAF,” “maximum VAF,” “max-MAF” or “MAX VAF” refer to the maximum or largest MAF of all somatic variants present or observed in a given sample.

Mutant Allele Frequency: As used herein, “mutant allele frequency,” “variant allele frequency,” “mutant allele fraction,” “variant allele fraction,” “MAF,” or “VAF” refers to the frequency at which mutant alleles occur in a given population of nucleic acids, such as a sample obtained from a subject. MAF is generally expressed as a fraction or a percentage.

Also it is understood by one of skill that response refers to a change in one or more circulating tumor DNA (ctDNA) variant allele frequencies, levels, or amounts observed in between samples taken from a given subject at different time points.

Molecular Responder: As used herein, “molecular responder” or “responder” refers to a subject having a molecular response score that indicates a decrease in one or more circulating tumor DNA (ctDNA) variant allele frequencies, levels, or amounts observed in between samples taken from the subject at different time points.

### II. Molecular Response Scoring

In an embodiment, a method for determining a Molecular response (MR) score is disclosed. The methods of this disclosure may have a wide variety of uses in the manipulation, preparation, identification, quantification, and/or analysis of cell-free nucleic acids. Molecular response is an assessment of the change in circulating tumor DNA (ctDNA) load on-treatment (usually 3-10 weeks) in comparison to pre-treatment baseline. Molecular response is associated with patient response to therapy and long term outcomes across solid tumors and therapy types. Molecular response can also be used to predict clinical response earlier than radiographic and/or RECIST response. Multiple methods have been used to calculate molecular response and there is no consensus regarding which method is best.

Methods and systems are described for assessing response to treatment using a molecular response (MR) score. In an embodiment, baseline (pre-treatment) gene expression data may be obtained for a plurality of patients prior to treatment and on-treatment gene expression data may be obtained for the plurality of patients during treatment. In an embodiment, the baseline gene expression data (e.g., variant data) and/or the on-treatment gene expression data may be analyzed to determine a molecular response (MR) score. The MR score may indicate that a patient is a responder or a non-responder to the treatment. In an embodiment, a mutant allele fraction (MAF) may be determined as part of the MR score. In an embodiment, the variance of each MAF may be incorporated into the determination of the molecular response score. This ensures molecular response scores include accurate variance, which provides a significant improvement in making a correct conclusion from the molecular response score. The improvement is even more pronounced when the molecular response score is a ratio, as a ratio is sensitive to variance in the denominator. The variance can be incorporated into the molecular response score either through deriving mathematically the molecular response variance or through simulation or sampling from the variance distribution of each variant to determine the molecular response variance.

A. cfDNA Isolation and Extraction

At a first time T₀, baseline cfDNA may be obtained from one or more baseline samples obtained from one or more subjects prior to treatment at step 101 and at a second time T₁, on-treatment cfDNA may be obtained from one or more on-treatment samples obtained from one or more subjects after treatment at step 102. Treatment may occur/being at any time subsequent to time T₀. For example, treatment may occur minutes, hours, days, etc. after time T₀. By way of further example, treatment may occur 30 minutes after time T₀, 1 hour to 2 hours after time T₀, 1 day to 2 days after time T₀, 1 week to 2 weeks after time T₀, 1 month to 2 months after time T₀, 6 months to 1 year after time T₀, 1 year to 2 years after time T₀, and the like. Time T₁ can be any amount of time after time T₀, for example, any time between and including 1-24 hours, 1-180 days, 1-12 weeks, 6-12 months, and the like.

As described herein, a polynucleotide can comprise any type of nucleic acid, such as DNA and/or RNA. For example, if a polynucleotide is DNA, it can be genomic DNA, complementary DNA (cDNA), or any other deoxyribonucleic acid. A polynucleotide can also be a cell-free nucleic acid such as cell-free DNA (cfDNA). For example, the polynucleotide can be circulating cfDNA. Circulating cfDNA may comprise DNA shed from bodily cells via apoptosis or necrosis. cfDNA shed via apoptosis or necrosis may originate from normal (e.g. healthy) bodily cells. Where there is abnormal tissue growth, such as for cancer, tumor DNA may be shed. The circulating cfDNA can comprise circulating tumor DNA (ctDNA).

i. Samples

Isolation and extraction of cell free polynucleotides may be performed through collection of samples using a variety of techniques. A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled blood is typically between about 5 ml to about 20 ml.

The sample can comprise various amounts of nucleic acid. Typically, the amount of nucleic acid in a given sample is equated with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations). In some embodiments of the present disclosure, cell free nucleic acids in a subject may derive from a tumor. For example cell-free DNA isolated from a subject can comprise ctDNA.

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps. Additional details regarding cfDNA partitioning and related analysis of epigenetic modifications that are optionally adapted for use in performing the methods disclosed herein are described in, for example, WO 2018/119452, filed Dec. 22, 2017, which is incorporated by reference.

ii. Nucleic Acid Tags

In certain embodiments, tags providing molecular identifiers or barcodes are incorporated into or otherwise joined to adapters by chemical synthesis, ligation, or overlap extension PCR, among other methods. In some embodiments, the assignment of unique or non-unique identifiers, or molecular barcodes in reactions follows methods and utilizes systems described in, for example, U.S. patent applications 20010053519, 20030152490, 20110160078, and U.S. Pat. Nos. 6,582,908, 7,537,898, and 9,598,731, which are each incorporated by reference.

Tags are linked (e.g., ligated) to sample nucleic acids randomly or non-randomly. In some embodiments, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some embodiments, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain embodiments, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique or non-unique.

One exemplary format uses from about 2 to about 1,000,000 different tags, or from about 5 to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

In some embodiments, identifiers are predetermined, random, or semi-random sequence oligonucleotides. In other embodiments, a plurality of barcodes may be used such that barcodes are not necessarily unique to one another in the plurality. In these embodiments, barcodes are generally attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) portions of sequence reads typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

iii. Nucleic Acid Amplification

Sample nucleic acids flanked by adapters are typically amplified by PCR and other amplification methods using nucleic acid primers binding to primer binding sites in adapters flanking a DNA molecule to be amplified. In some embodiments, amplification methods involve cycles of extension, denaturation and annealing resulting from thermocycling, or can be isothermal as, for example, in transcription mediated amplification. Other exemplary amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce sample indexes/tags to a nucleic acid molecule using conventional nucleic acid amplification methods. The amplifications are typically conducted in one or more reaction mixtures. In some embodiments, molecular tags and sample indexes/tags are introduced prior to and/or after sequence capturing steps are performed. In some embodiments, only the molecular tags are introduced prior to probe capturing and the sample indexes/tags are introduced after sequence capturing steps are performed. In certain embodiments, both the molecular tags and the sample indexes/tags are introduced prior to performing probe-based capturing steps. In some embodiments, the sample indexes/tags are introduced after sequence capturing steps (i.e., enrichment of nucleic acids) are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

iv. Nucleic Acid Enrichment

In some embodiments, sequences are enriched prior to sequencing the nucleic acids. Enrichment is optionally performed for specific target regions or nonspecifically (“target sequences”). By way of example, enrichment may be performed nonspecifically based on a size selection method that is not sequence specific but rather is sequence fragment size specific. In some embodiments, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some embodiments, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain embodiments, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

b. Nucleic Acid Sequencing

After extraction and isolation of cfDNA from samples, the cfDNA may be sequenced. Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfite sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, primer walking, sequencing using PacBio, SOLID, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one more nucleic acid fragment types or sections known to contain markers of cancer or of other diseases. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some embodiments, cell-free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An exemplary read depth is from about 1000 to about 50000 reads per locus (base position).

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U). Exemplary enzymes or catalytic fragments thereof that are optionally used include Klenow large fragment and T4 polymerase. At 5′ overhangs, the enzyme typically extends the recessed 3′ end on the opposing strand until it is flush with 5′ end to produce a blunt end. At 3′ overhangs, the enzyme generally digests from 3′ end up to and sometimes beyond 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a template/parent nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence may be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, for example, Levy et al., Annual Review of Genomics and Human Genetics, 17:95-115 (2016), Liu et al., J. of Biomedicine and Biotechnology, Volume 2012, Article ID 251364:1-11 (2012), Voelkerding et al., Clinical Chem., 55:641-658 (2009), MacLean et al., Nature Rev. Microbiol., 7:287-296 (2009), Astier et al., J Am Chem Soc., 128 (5): 1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

i. Sequencing Panel

To improve the likelihood of detecting genomic regions of interest and optionally, tumor indicating mutations, the sections of DNA sequenced may comprise a panel of genes or genomic sections that comprise known genomic regions. Selection of a limited section for sequencing (e.g., a limited panel) can reduce the total sequencing needed (e.g., a total amount of nucleotides sequenced). A sequencing panel can target a plurality of different genes or regions, for example, to detect a single cancer, a set of cancers, or all cancers. Alternatively, DNA may be sequenced by whole genome sequencing (WGS) or other unbiased sequencing method without the use of a sequencing panel. Examples of suitable panel and targets for use in panels can be found in the epigenetic targets described in U.S. provisional patent application 62/799,637, filed Jan. 31, 2019, which is incorporated by reference in its entirety.

In some aspects, a panel that targets a plurality of different genes or genomic regions (e.g., transcriptional factor binding regions, distal regulatory elements (DREs), repetitive elements, intron-exon junctions, transcriptional start sites (TSSs), and/or the like) is selected such that a determined proportion of subjects having a cancer exhibits a genetic variant or tumor marker in one or more different genes in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some embodiments, markers for a tissue of origin are tissue-specific epigenetic markers.

Some examples of listings of genomic locations of interest may be found in Table 1 and Table 2. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or 97 of the genes of Table 1. In an embodiment, genomic locations used in the methods of the present disclosure comprise all genes of Table 1. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, or 70 of the SNVs of Table 1. In an embodiment, genomic locations used in the methods of the present disclosure comprise all SNVs of Table 1. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 1. In an embodiment, genomic locations used in the methods of the present disclosure comprise all CNVs of Table 1. In some embodiments, genomic locations used in the methods of the present disclosure comprise at least 1, at least 2, at least 3, at least 4, at least 5, or 6 of the fusions of Table 1. In an embodiment, genomic locations used in the methods of the present disclosure comprise all fusions of Table 1. In some embodiments, genomic locations used in the methods of the present disclosure comprise at least a portion of at least 1, at least 2, or 3 of the indels of Table 1. In an embodiment, genomic locations used in the methods of the present disclosure comprise all indels of Table 1. In an embodiment, genomic locations used in the methods of the present disclosure comprise all genes, SNVs, CNVs, fusions, and indels of Table 1. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or 115 of the genes of Table 2. In an embodiment, genomic locations used in the methods of the present disclosure comprise all genes of Table 2. In an embodiment, genomic locations used in the methods of the present disclosure comprise all genes of Table 1 and Table 2. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, at least 70, or 73 of the SNVs of Table 2. In an embodiment, genomic locations used in the methods of the present disclosure comprise all SNVs of Table 2. In an embodiment, genomic locations used in the methods of the present disclosure comprise all SNVs of Table 1 and Table 2. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 2. In an embodiment, genomic locations used in the methods of the present disclosure comprise all CNVs of Table 2. In an embodiment, genomic locations used in the methods of the present disclosure comprise all CNVs of Table 1 and Table 2. In some embodiments, genomic locations used in the methods of the present disclosure comprise at least 1, at least 2, at least 3, at least 4, at least 5, or 6 of the fusions of Table 2. In an embodiment, genomic locations used in the methods of the present disclosure comprise all fusions of Table 2. In an embodiment, genomic locations used in the methods of the present disclosure comprise all fusions of Table 1 and Table 2. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 2. In an embodiment, genomic locations used in the methods of the present disclosure comprise all indels of Table 2. In an embodiment, genomic locations used in the methods of the present disclosure comprise all indels of Table 1 and Table 2. In an embodiment, genomic locations used in the methods of the present disclosure comprise all genes, SNVs, CNVs, fusions, and indels of Table 2. In an embodiment, genomic locations used in the methods of the present disclosure comprise all genes, SNVs, CNVs, fusions, and indels of Table 1 and Table 2. Each of these genomic locations of interest may be identified as a backbone region or hot-spot region for a given bait set panel.

TABLE 1
SNVs, CNVs, fusions and indels

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | CDKN2B | CCNE1 | CDK4 | FGFR3 | 19 & |
| | | | | | | | | | 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & |
| | | | | | | | | | 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon |
| NPM1 | NRAS | NTRK1 | PDGFRA | PIK3CA | PTEN | PIK3CA | RAF1 | | 14 |
| | | | | | | | | | skipping) |
| PTPN11 | RAF1 | RB1 | RET | RHEB | RHOA | | | | |
| RIT1 | ROS1 | SMAD4 | SMO | SRC | STK11 | | | | |
| TERT | TP53 | TSC1 | VHL | | | | | | |

TABLE 2
SNVs, CNVs, fusions and indels

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | DDR2 | CCNE1 | CDK4 | FGFR3 | 19 & |
| | | | | | | | | | 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & |
| | | | | | | | | | 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon |
| NPM1 | NRAS | NTRK1 | PDGFRA | PIK3CA | PTEN | PIK3CA | RAF1 | | 14 |
| | | | | | | | | | skipping) |
| PTPN11 | RAF1 | RB1 | RET | RHEB | RHOA | | | | ATM |
| RIT1 | ROS1 | SMAD4 | SMO | MAPK1 | STK11 | | | | |
| TERT | TP53 | TSC1 | VHL | MAPK3 | MTOR | | | | |
| NTRK3 | | | | | | | | | APC |
| | | | | | | | | | ARID1A |
| | | | | | | | | | BRCA1 |
| | | | | | | | | | BRCA2 |
| | | | | | | | | | CDH1 |
| | | | | | | | | | CDKN2A |
| | | | | | | | | | GATA3 |
| | | | | | | | | | KIT |
| | | | | | | | | | MLH1 |
| | | | | | | | | | MTOR |
| | | | | | | | | | NF1 |
| | | | | | | | | | PDGFRA |
| | | | | | | | | | PTEN |
| | | | | | | | | | RB1 |
| | | | | | | | | | SMAD4 |
| | | | | | | | | | STK11 |
| | | | | | | | | | TP53 |
| | | | | | | | | | TSC1 |
| | | | | | | | | | VHL |

In some embodiments, the one or more regions in the panel comprise one or more loci from one or a plurality of genes for detecting residual cancer after surgery. This detection can be earlier than is possible for existing methods of cancer detection. In some embodiments, the one or more genomic locations in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some embodiments, the methods described herein detect the response of patients to cancer therapy (particularly in high risk patients) earlier than is possible for existing methods of cancer detection.

A genomic location may be selected for inclusion in a sequencing panel based on a number of subjects with a cancer that have a tumor marker in that gene or region. A genomic location may be selected for inclusion in a sequencing panel based on prevalence of subjects with a cancer and a tumor marker present in that gene. Presence of a tumor marker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A database may comprise information about regulatory elements or genomic regions in tumor samples. The information relating to the sequenced tumor samples may include the frequency of various genetic variants and describe the genes or regions in which the genetic variants occur. The genetic variants may be tumor markers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with tumor marker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or genomic section may be selected for a panel where the frequency of a tumor marker is significantly greater in sampled tumor tissue or circulating tumor DNA than found in a given background population. A combination of genomic locations may be selected for inclusion of a panel such that at least a majority of subjects having a cancer may have a tumor marker or genomic region present in at least one of the genomic location or genes in the panel. The combination of genomic location may be selected based on data indicating that, for a particular cancer or set of cancers, a majority of subjects have one or more tumor markers in one or more of the selected regions. For example, to detect cancer 1, a panel comprising regions A, B, C, and/or D may be selected based on data indicating that 90% of subjects with cancer 1 have a tumor marker in regions A, B, C, and/or D of the panel. Alternately, tumor markers may be shown to occur independently in two or more regions in subjects having a cancer such that, combined, a tumor marker in the two or more regions is present in a majority of a population of subjects having a cancer. For example, to detect cancer 2, a panel comprising regions X, Y, and Z may be selected based on data indicating that 90% of subjects have a tumor marker in one or more regions, and in 30% of such subjects a tumor marker is detected only in region X, while tumor markers are detected only in regions Y and/or Z for the remainder of the subjects for whom a tumor marker was detected. Tumor markers present in one or more genomic locations previously shown to be associated with one or more cancers may be indicative of or predictive of a subject having cancer if a tumor marker is detected in one or more of those regions 50% or more of the time. Computational approaches such as models employing conditional probabilities of detecting cancer given a cancer frequency for a set of tumor markers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, bisulfate sequencing, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the mutant allele frequency for a given genetic variant. The mutant allele frequency may refer to the frequency at which mutant alleles (e.g., not the most common allele) occurs in a given population of nucleic acids, such as a sample. Genetic variants at a low mutant allele frequency may have a relatively low frequency of presence in a sample. In some cases, the panel allows for detection of genetic variants at a mutant allele frequency of at least 0.0001%, 0.001%, 0.005%, 0.01%, 0.05%, 0.1%, or 0.5%. The panel can allow for detection of genetic variants at a mutant allele frequency of 0.001% or greater. The panel can allow for detection of genetic variants at a mutant allele frequency of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of tumor markers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of tumor markers at a frequency in a sample as low as 1.0%. The panel can allow for detection of tumor markers at a frequency in a sample as low as 0.75%. The panel can allow for detection of tumor markers at a frequency in a sample as low as 0.5%. The panel can allow for detection of tumor markers at a frequency in a sample as low as 0.25%. The panel can allow for detection of tumor markers at a frequency in a sample as low as 0.1%. The panel can allow for detection of tumor markers at a frequency in a sample as low as 0.075%. The panel can allow for detection of tumor markers at a frequency in a sample as low as 0.05%. The panel can allow for detection of tumor markers at a frequency in a sample as low as 0.025%. The panel can allow for detection of tumor markers at a frequency in a sample as low as 0.01%. The panel can allow for detection of tumor markers at a frequency in a sample as low as 0.005%. The panel can allow for detection of tumor markers at a frequency in a sample as low as 0.001%. The panel can allow for detection of tumor markers at a frequency in a sample as low as 0.0001%. The panel can allow for detection of tumor markers in sequenced cfDNA at a frequency in a sample as low as 1.0% to 0.0001%. The panel can allow for detection of tumor markers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the genomic positions in the panel.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The locations comprising genomic regions in the panel can be selected so that one or more epigenetically modified regions are detected. The one or more epigenetically modified regions can be acetylated, methylated, ubiquitylated, phosphorylated, sumoylated, ribosylated, and/or citrullinated. For example, the regions in the panel can be selected so that one or more methylated regions are detected.

The regions in the panel can be selected so that they comprise sequences differentially transcribed across one or more tissues. In some cases, the locations comprising genomic regions can comprise sequences transcribed in certain tissues at a higher level compared to other tissues. For example, the locations comprising genomic regions can comprise sequences transcribed in certain tissues but not in other tissues.

The genomic locations in the panel can comprise coding and/or non-coding sequences. For example, the genomic locations in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the genomic locations in the panel can comprise sequences in non-coding RNA, e.g., ribosomal RNA, transfer RNA, Piwi-interacting RNA, and microRNA.

The genomic locations in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the cancer with a sensitivity of 100%.

The genomic locations in the panel can be selected to detect (diagnose) a cancer with a desired level of specificity (e.g., through the detection of one or more genetic variants). For example, the genomic locations in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the one or more genetic variant with a specificity of 100%.

The genomic locations in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, genomic locations in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The genomic locations in the panel can be selected to detect (diagnose) a cancer with a desired accuracy. As used herein, the term “accuracy” may refer to the ability of a test to discriminate between a disease condition (e.g., cancer) and healthy condition. Accuracy may be can be quantified using measures such as sensitivity and specificity, predictive values, likelihood ratios, the area under the ROC curve, Youden's index and/or diagnostic odds ratio.

Accuracy may be presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The genomic locations in the panel can be selected to detect cancer with an accuracy of 100%.

A panel may be selected to be highly sensitive and detect low frequency genetic variants. For instance, a panel may be selected such that a genetic variant or tumor marker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a sensitivity of 70% or greater. A panel may be selected to detect a tumor marker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or tumor marker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a specificity of 70% or greater. A panel may be selected to detect a tumor marker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or tumor marker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with an accuracy of 70% or greater. A panel may be selected to detect a tumor marker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or tumor marker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

In an embodiment, after sequencing, sequence reads may be assigned a quality score. A quality score may be a representation of sequence reads that indicates whether those sequence reads may be useful in subsequent analysis based on a threshold. In some cases, some sequence reads are not of sufficient quality or length to perform a subsequent mapping step. Sequence reads with a quality score at least 90%, 95%, 99%, 99.9%, 99.99% or 99.999% may be filtered out of a data set of sequence reads. In other cases, sequence reads assigned a quality scored at least 90%, 95%, 99%, 99.9%, 99.99% or 99.999% may be filtered out of the data set. Sequence reads that meet a specified quality score threshold may be mapped to a reference genome. After mapping alignment, sequence reads may be assigned a mapping score. A mapping score may be a representation of sequence reads mapped back to the reference sequence indicating whether each position is or is not uniquely mappable. Sequence reads with a mapping score at least 90%, 95%, 99%, 99.9%, 99.99% or 99.999% may be filtered out of the data set. In other cases, sequencing reads assigned a mapping scored less than 90%, 95%, 99%, 99.9%, 99.99% or 99.999% may be filtered out of the data set.

c. MAF Determination

After cfDNA sequencing of samples, one or more mutant allele fractions (MAFs) may be determined. Some or all MAF determination may occur prior to variant classification, after variant classification, during variant classification, before variant filtering, after variant filtering, during variant filtering, or a combination thereof. Prior to step, cfDNA can be end repaired, ligated with adapters comprising molecular barcodes, amplified, and enriched. Amplification can incorporate sample index. In an embodiment, MAF values may be determined for all variants or all somatic variants. In an embodiment, MAF values may be determined for less than all variants or less than all somatic variants. Variant allele fraction (VAF) is used herein interchangeably with MAF. The mutant allele fraction (MAF) represents the number of mutant molecules divided by the total number of molecules (e.g., molecular coverage) at a specific genomic position:

$$MAF = \frac{Number of mutant molecules}{Total number of molecules}$$

A maximum MAF may be determined as the maximum or largest MAF of all somatic variants present or observed in a given sample. In some embodiments, maximum MAF can be considered as tumor fraction of a given sample.

A maximum fraction of diploid genes (“max frac_diploid”) (least allele imbalance) may be determined. A fraction of diploid genes (“frac_diploid) is a measure of the level of allele imbalance across the sample as determined by copy number. Samples with high levels of allele imbalance are prone to germline/somatic misclassification. Therefore, a low level of allele imbalance (or high frac_diploid) is an indication of the reliability of the somatic classification call.

In an embodiment, a total coverage profile may be used to capture fold change and thus tumor fraction, rather than individual genes.

d. Variant Classification

Sequencing at steps 103 and 104 generates a plurality of sequence reads. The plurality of sequence reads may be analyzed to determine one or more variants and to classify the one or more variants at steps 107 and/or 108. In an embodiment, some or all variant classification may be determined prior to MAF determination 105/106, after MAF determination 105/106, during MAF determination 105/106, or combinations thereof. Variants may include, for example, single nucleotide variants (SNV's), indels, fusions, and copy number variation. Any known technique for variant calling may be used. In an embodiment, the plurality of sequence reads from a sample may be assembled and/or mapped and aligned to genomic positions relative to a reference genome. In some embodiments, the plurality of sequence reads (assembled or otherwise) may then be compared to the reference genome to determine how the plurality of sequence reads of the subject vary from that of the reference genome. Such a process may determine the presence of one or more variants in the plurality of sequence reads. In some embodiments, the molecular barcodes and/or start and stop genomic positions of a nucleic acid molecule obtained from the plurality of sequence reads can be used to identify the mutant molecules where the sequence reads belonging to the molecule differ from the reference genome. Such a process may determine the presence of one or more variants in the plurality of sequence reads.

In an embodiment, common heterozygous SNPs may be used to model local germline allele count behavior and call variants somatic if they deviate significantly from observed germline mutant allele fraction. A betabinomial model may be used as it models both the mean and variance of mutant allele counts at common SNPs. For example, the betabinomial model described in PCT/US2018/052087, hereby incorporated by reference in its entirety, can be used. This is an improvement over simpler methods like fixed MAF cutoffs or Poisson models as they may not represent the variance in molecule counts appropriately.

e. Variant Filtering

In an embodiment, one or more filtering processes may be applied to the sequence reads to exclude sequence reads from further analysis. In an embodiment, some or all filtering may be applied prior to MAF determination, after MAF determination during MAF determination, before variant classification, after variant classification, during variant classification, or a combination thereof.

In some embodiments, one or more somatic variants having MAFs that are less than about 0.1%, 0.2%, 0.3%, 0.4%, 0.5%, 0.6%, 0.7%, 0.8%, or 0.9% at the first and/or second time points may be excluded from further analysis. In some embodiments, one or more somatic variants having less than 5, 10, 15, 20, 25 or 30 mutant molecule counts at the first and/or second time points may be excluded from further analysis. In some embodiments, one or more somatic variants having a coverage less than 50, 100, 200, 300, 400, 500, 600, 700, 800, 900 or 1000 at the first and/or second time points may be excluded from further analysis.

In an embodiment, copy number variants may be used to exclude sequence reads from further analysis. Copy number amplifications may be determined as is known in the art. At step 109, the method 100 may filter out copy number amplifications in genes with either insufficient probe coverage or insufficient copy number (e.g. below the 95% limit of detection).

By way of example, CNVs may be determined by analyzing sequence reads to generate a chromosomal region of coverage. The chromosomal regions may be divided into variable length windows or bins. Read coverage may be determined for each window/bin region. In an embodiment, a quantitative measure related to sequencing read coverage is a measure indicative of the number of reads derived from a DNA molecule corresponding to a genetic locus (e.g., a particular position, base, region, gene or chromosome from a reference genome). In order to associate reads to a genetic locus, the reads can be mapped or aligned to the reference. Software to perform mapping or aligning (e.g., Bowtie, BWA, mrsFAST, BLAST, BLAT) can associate a sequencing read with a genetic locus. After the sequence read coverage has been determined, a stochastic modeling algorithm may be applied to convert the normalized nucleic acid sequence read coverage for each window/bin region to the discrete copy number states. In some cases, this algorithm may comprise one or more of the following: Hidden Markov Model, dynamic programming, support vector machine, Bayesian network, trellis decoding, Viterbi decoding, expectation maximization, Kalman filtering methodologies and neural networks. The discrete copy number states of each window region can be utilized to identify copy number variation in the chromosomal regions. In some cases, all adjacent window/bin regions with the same copy number can be merged into a segment to report the presence or absence of copy number variation state. In some cases, various windows/bins can be filtered before they are merged with other segments. Copy number variation may be used to report a percentage score indicating how much disease material (or nucleic acids having a copy number variation) exists in a cell free polynucleotide sample.

In an embodiment, the existence of CNVs in one or more genes may be used to exclude variants from further analysis. By way of example, variants having a threshold number of LDT-reportable genes with copy number >=a gene-specific 95% limit of detection (LoD) in either T₀ or T₁ sample. The threshold may be from about 10 to about 30. The threshold may be, for example, 15, 16, 17, 18, 19, 20, 21, 22, 23, 24, 25, etc. In an embodiment, the threshold may be 19.

Copy number variation may indicate fold change for a given variant. A Gaussian model may be used to determine a ratio of fold changes between time T₀ and time T₁ which may be used as an estimate of molecular response score.

In an embodiment, in the event the subject has no somatic variants, or has no variants that satisfy criteria of the variant filtering process, the subject may be classified as not-evaluable. In an embodiment, a subject classified as non-evaluable may be further classified as a molecular responder. In an embodiment, a subject having low ctDNA at both time T₀ and time T₁ may be classified as non-evaluable and further classified as a molecular responder. In an embodiment, a subject having a low MAF at both time T₀ and time T₁ may be classified as non-evaluable and further classified as a molecular responder. In an embodiment, a subject having a low tumor fraction at both time T₀ and time T₁ may be classified as non-evaluable and further classified as a molecular responder. Low MAF or low tumor fraction may refer to an MAF or a tumor fraction below a limit of detection (e.g. below the 95% limit of detection), or below a limit of quantification. What constitutes low may depend on panel design, but for example, an MAF f 0.1, 0.2, or 0.3% may be considered low.

i. Germline Filter

In an embodiment, a germline filter may be applied to the sequence reads. Some (e.g., less than all) or all steps shown may be performed in any combination and in any order. Samples collected over the course of a subject's treatment (e.g., samples collected at time T₀ and at time T₁) may have differing levels of tumor shedding and allele imbalance, meaning that variant classification at step 107/108 may be prone to assign differing somatic classifications for the same variant in the same subject. Since the aim of molecular response is to track the somatic variants over the course of treatment, a classification discrepancy may be automatically resolved to properly remove germline variants from consideration by reclassifying variants. For example, a variant may be classified as somatic at time T₀ and germline at time T₁. For example, a variant may be classified as germline at time T₀ and somatic at time T₁. For example, a variant may be classified as germline at time T₀ and not classified at time T₁. For example, a variant may be classified as somatic at time T₀ and not classified at time T₁. The germline filter 200 is configured to resolve such discrepancies and reassign variant classification.

As shown, a determination may be made for at least one variant in the sequence reads as to whether the variant is a deleterious variant (e.g., a frameshift or nonsense mutation) in a tumor suppressing gene (TSG). For example, the variant may be compared to a database of known TSG's. If the variant is a deleterious variant in a TSG, the variant may be classified as somatic, regardless of the classification result (e.g., the classification will be changed from germline to somatic).

If the variant is not a deleterious variant in a TSG, the germline filter may determine the maximum MAF of variants present in a sample and the maximum fraction of diploid genes for at least one variant in the sample. If the maximum fraction of diploid genes for a variant (in one of the at least two time points) indicates that the variant is somatic and the MAF for the variant (in one of the at least two time points) does not increase the maximum MAF, the variant may be classified as somatic, regardless of the classification result (e.g., the classification will be changed from germline to somatic). If, the maximum fraction of diploid genes for a variant (in one of the at least two time points) indicates that the variant is germline and the MAF for the variant (in one of the at least two time points) would increase the maximum MAF, the variant may be classified as germline, regardless of the classification result (e.g., the classification will be changed from somatic to germline).

If, the maximum fraction of diploid genes for the variant indicates that the variant is somatic and the MAF for the variant would increase the maximum MAF—or—if the maximum fraction of diploid genes for the variant indicates that the variant is germline and the MAF for the variant would not increase the maximum MAF, the germline filter, may determine if the variant is classified as somatic in another patient sample at less than a threshold percentage (in one of the at least two time points). The threshold percentage may be at least about 1%, 2%, 3%, 4%, 5%, 6%, 7%, 8%, or 9%. If the variant is classified as somatic in another patient sample at less than the threshold percentage the variant may be classified as somatic, regardless of the classification result at step 107/108 (e.g., the classification will be changed from germline to somatic).

If, the variant is not classified as somatic in another patient sample at <5%, the germline filter may determine if the MAF for a variant (in one of the at least two time points) is larger than another MAF in the sample. For example, the germline filter may determine if the MAF for the variant is at least about two times greater, three times greater, four times greater, five times greater, six times greater, seven times greater, eight times greater, nine times greater, or at least 10 times greater than one or more other MAFs in the same sample. The one or more other MAFs in the sample may be, for example, the next highest somatic MAF to the max MAF in the sample. If the MAF for the variant is larger than another MAF in the sample the variant may be classified as germline, regardless of the classification result (e.g., the classification will be changed from somatic to germline).

The germline filter may determine if the MAF for a variant (in one of the at least two time points) is larger than another MAF in another sample. For example, the germline filter may determine if the MAF for the variant is at least about two times greater, three times greater, four times greater, five times greater, six times greater, seven times greater, eight times greater, nine times greater, or at least 10 times greater than one or more other MAFs in another sample. The one or more other MAFs in another sample may be, for example, the max MAF of the other sample. If the MAF for the variant is larger than another MAF in another sample the variant may be classified as germline, regardless of the classification result (e.g., the classification will be changed from somatic to germline).

If, the MAF for the variant is neither larger than another MAF in the sample nor larger than another MAF in another sample, the germline filter may classify the variant as germline, regardless of the classification result (e.g., the classification will be changed from somatic to germline).

Those variants classified as germline may be excluded from further analysis, including for example, MAF determination and/or MR scoring. In some embodiments, variants are classified as CHIP variants when those variants are classified as CHIP in at least one patient sample.

ii. CHIP Filter

cfDNA can comprise an aggregate of cfDNA from any cell types including tumor, blood cell and the like. Clonal hematopoiesis of intermediate potential mutation (CHIP) may even be present in cfDNA. Common approaches for CHIP filtering leverages recurrent CHIP genes or hotspots curated by large public or internal cohort studies. However, these approaches do not address challenges in identifying random CHIP mutations in a plasma only approach. Residual unfiltered CHIP variants would bias the fractional change towards 1 (unchanged) and thus yield inaccurate subsequent molecular response prediction. To filter private CHIP variants (e.g., a variant that is CHIP but has not been documented ever or not often in previous databases of known CHIP variants), mutation measurement between two timepoints can be used to cluster variants of similar fractional change. When a patient receive treatment, progression or response will result in fractional somatic mutation while CHIP variant will remain stable. By clustering mutations into clones, random CHIP variants can be found in clones with enrichment of known CHIP list or in clones with stable fractional difference.

Accordingly, provided herein is an improvement in CHIP filtering that leverages the observations between two timepoints (T₀ and T₁) to cluster genomic mutations in clones with differing fractional change. CHIP filtering may group/cluster events into clones to estimate % clone load change. The clustering procedure may start with each single event and then merge utilizing a novel clustering heuristic. Once the % clone load change is determined using all the events, each clone can be inspected based on composition of variants and % clone load change to determine if the variant is a CHIP clone.

In one embodiment, the genomic mutations/variants are clustered utilizing a novel agglomerative hierarchical clustering heuristic. The heuristic quantifies the statistical dissimilarity between mutations/variants and clusters via a custom dissimilarity metric. A tunable stopping rule is utilized which continues agglomeration until a minimum (or maximum, depending upon the metric) allowable dissimilarity threshold is met. In one embodiment, the custom dissimilarity metric is a modification of the Bhattacharyya distance such that a numerical integration is performed with respect to the product (not subjected to a square root) of the scaled likelihoods of the mutations/variants and/or clusters that are under consideration to be merged at a given step of the clustering heuristic. The likelihoods are scaled to numerically integrate to 1 over the support of the integration. For SNVs and indels, the likelihood is calculated with respect to a Beta-Binomial model approximation of the observed count data that informs the MAF determination for the variants being clustered. The dispersion of the Beta-Binomial model is set via a tunable parameter. For CNVs, the likelihood is calculated with respect to a Gaussian model approximation of the observed fold change estimates of the mutations of interest, with the variability of Gaussian model also set via a tunable parameter. The agglomeration of mutations is conducted in a novel fashion such that, in some instances, clustering is performed via a tiered approach, in which a first set of mutations is clustered until the stopping rule is met and then a second set of mutations is introduced and further agglomerative steps are possibly performed according to the same dissimilarity metric and stopping rule. In some circumstances, a third set of mutations is introduced in a similar manner following the application of the clustering heuristic to the second set of mutations.

In an embodiment, a CHIP filter may estimate, a scaled likelihood function Pᵢ(Rᵢ) for each mutation/variant in a sample, where i=1, . . . , Iₘᵥ is the index for each unique qualifying mutation/variants observed across the two time points for a given sample, assuming a total of Iₘᵥ qualifying mutation/variants are observed. For ease of presentation, we denote the number of observed mutation/variant counts at time point 1 for the i^{th} mutation/variant as

$${m}_{i : 1}^{obs} ,$$

and the total number of counts at the genomic location and at time point 1 as

$${n}_{i : 1}^{obs} .$$

Similarly, define

$${m}_{i : 2}^{obs} and {n}_{i : 2}^{obs} ,$$

but for time point 2. Define

$${v}_{i : 1}^{true} and {v}_{i : 2}^{true}$$

as the true mutation/variant allele fractions as time points 1 and 2, respectively. The heuristic is designed to estimate

$${R}_{i} = {v}_{i : 2}^{true} / {v}_{i : 1}^{true}$$

and then to cluster together mutations/variants with Rᵢ values that can be plausibly considered to be identical. One embodiment of the heuristic is as follows:

| | | Unrestricted | Model Restricted |
|---|---|---|---|
| | True Variant | Estimates of Variant | Estimates of Variant |
| Time | Allele Fraction | Allele Fraction | Allele Fraction |
| T₀ | υ_{i:1}^{true} | v_{i:1}^{obs} = m_{i:1}^{obs}/n_{i:1}^{obs} | υ_{i:1} = v_{i:1}^{obs} |
| T₁ | υ_{i:2}^{true} | v_{i:2}^{obs} = m_{i:2}^{obs}/n_{i:2}^{obs} | υ_{i:2} = rᵢv_{i:1}^{obs} |

Pᵢ(Rᵢ) may be determined as:

$${P}_{i} ( {R}_{i} = {r}_{i} ) = {c}_{i} {f}_{i : 1} ( {m}_{i : 1}^{obs} , {n}_{i : 1}^{obs} ) {f}_{i : 2} ( {m}_{i : 2}^{obs} , {n}_{i : 2}^{obs} , {m}_{i : 1}^{obs} , {n}_{i : 1}^{obs} , {r}_{i} )$$

$$where$$

$${f}_{i : 1} ( {m}_{i : 1}^{obs} , {n}_{i : 1}^{obs} ) ∼ Binomial ( x = {m}_{i : 1}^{obs} , N = {n}_{i : 1}^{obs} , p = {v}_{i : 1}^{obs} )$$

$$and$$

$${f}_{i : 2} ( {m}_{i : 2}^{obs} , {n}_{i : 2}^{obs} , {m}_{i : 1}^{obs} , {n}_{i : 1}^{obs} , {r}_{i} ) ∼ Binomial ( x = {m}_{i : 2}^{obs} , N = {n}_{i : 2}^{obs} , p = {r}_{i} {v}_{i : 1}^{obs} )$$

and
cᵢ is calculated such that the numeric integration of Pᵢ(Rᵢ=rᵢ) across a support of candidate rᵢ values is equal to 1. This example embodiment assumes that the data is not over-dispersed with respect to the Binomial model and corresponds to a special case of the more general class of Beta-Binomial models.

Approximate confidence intervals can be calculated for Rᵢ in a variety of ways, including via a highest density interval like approach in which the scaled likelihood for Pᵢ(Rᵢ=rᵢ) is considered to be an approximate posterior density estimate of Rᵢ assuming an improper prior distribution for the Rᵢ values.

The set of mutations/variants may be pairwise agglomerated according to Pᵢ(Rᵢ). For all possible pairings {i′, i*: i′≠i*; i′, i*=1, 2, . . . , Iₘᵥ} the dissimilarity measure, D(i′, i*), between P_{i′}(R_{i′}) and P_{i*}(R_{i*}) is calculated using a modified Bhattacharyya distance. Larger values of D(i′, i*) indicate that the mutation pair {i′, i*} are more likely to be realizations from the same underlying fractional change distribution. Accordingly, a pair of mutations/variants with the greatest value of D (⋅,⋅) may be merged into a single clone and Pᵢ(Rᵢ) for that clone may be updated. Pairwise agglomerations may continue until stopping criteria are satisfied or all mutations/variants have agglomerated to a single clone. The threshold may be and/or include values ranging from about 0.0005 to 0.005.

The number of clones and associated fractional change between timepoints may be reported with a confidence interval. Clones having a fractional change between the first and second time points at or above a predetermined threshold value may be identified. If multiple clones are identified, clones with a fractional change close to 1 and/or clones with specific known CHIP variants may be classified as potential CHIP variants. CHIP variants may be excluded from further analysis. In some embodiments, variants may be classified as CHIP variants when those variants are classified as CHIP in at least one patient sample.

Described herein is an example application of the CHIP filter, including an example of an agglomeration procedure. In the example, there are three qualified mutants identified. One can display scaled likelihood functions for each mutant (y-axis) over the support of R (x-axis). Suppose the mutant corresponding to a first likelihood of the scaled likelihood functions for each mutant is a known CHIP mutation. Mutants in the left panel with the most similarity are annotated with stars. The middle panel displays the resulting agglomerated likelihood from the merging of the first likelihood and a second likelihood the scaled likelihood functions for clones in the left panel. A third likelihood of the scaled likelihood functions for each clone from the left panel has a likelihood function that is unaltered by the agglomeration. The right panel displays the final clonality. Since the composition of the second likelihood clone is 50% CHIP, the second likelihood one may be identified as putatively CHIP. This would result in the final value of R being defined solely by the third likelihood clone.

Described herein is a method that includes determining a tumor load change (R) for tumor fraction change P(R) for each of a plurality of variants from sequence information generated from targeted nucleic acids associated with one or more cancer types in samples obtained from the subject at first and second time points to produce a set of tumor load changes. In addition, method also includes identifying one or more resistance signatures corresponding to one or more clonal variants from the set of tumor load changes.

f. MR Score

The method may proceed to determine an MR score at step. In an embodiment, the MR score may be determined using MAF values associated with somatic variants remaining after variant filtering. In an embodiment, MAF values of all the somatic variants may be used. In an embodiment, MAF values of less than all the somatic variants may be used. As described at step, MAFs may be determined for a plurality of somatic variants from sequence reads generated from targeted nucleic acids associated with one or more cancer types in samples obtained from the subject at T₀ (e.g., pre-treatment) and T₁ (e.g., on-treatment) to produce sets of first and second MAFs for somatic variants in the plurality of somatic variants. An MR score can be expressed as a fraction or as a percentage. An MR score may be determined according to a method. The method may comprise determining a ratio of the first MAFs and second MAFs for somatic variants in the plurality of somatic variants to produce a set of MAF ratios and a corresponding standard deviation for an MAF ratio in the set of MAF ratios at step 601. In some embodiments, the standard deviation can be utilized as a criterion for reporting the MR score. For example, the standard deviation of the MR score, based on the individual standard deviations of at least one variant, can be used to determine a confidence interval and a subsequent cutoff for sample evaluability. In some embodiments, the cutoff can be at least 0.1, 0.15, 0.2, 0.3, 0.4 or 0.5. At step 602, for a subject, a weighted mean of the MAF ratios may be determined using the formula:

$$\frac{\sum ( weight * ratio )}{\sum weights}$$

where weight is 1/range\hat{ }2 for a given somatic variant in the plurality of somatic variants, where range is a difference between values of the first and second MAFs for a given somatic variant in the plurality of somatic variants, and ratio is a given MAF ratio in the set of MAF ratios. A confidence interval may be determined using the formula:
weighted mean of the MAF ratios+/−\sqrt{ratio variance},
where ratio variance is

$$\frac{1}{\sum weights} .$$

In an embodiment, in addition to, or as an alternative to, the weighted mean of MAF ratios as an MR score, a method is disclosed that clusters variants based on MAF ratios, calculates an aggregate MAF ratio for the cluster, and then uses as the MR score either a single selected cluster ratio or the weighted mean of the cluster ratios. The clustering may be performed by combining pairs of variants with overlapping MAF ratio distributions, or other clustering methods. The single selected cluster may be that which contains a known cancer driver variant, or absence of known clonal hematopoiesis variants. Cluster weights may also depend on the presence of a known cancer driver variant or the maximum VAF or number of variants in the cluster.

A, an MR score may be determined by a weighted mean of the first MAFs and a weighted mean of the second MAFs for a somatic variant in the plurality of somatic variants and a corresponding standard deviation for a weighted MAF ratio at. In some embodiments, the standard deviation can be utilized as a criterion for reporting the MR score. For example, the standard deviation of the MR score, based on the individual standard deviations of at least one variant, can be used to determine a confidence interval and a subsequent cutoff for sample evaluability. In some embodiments, the cutoff can be at least 0.1, 0.15, 0.2, 0.3, 0.4 or 0.5. At step, for a subject, a ratio of the weighted means of the MAFs may be determined. A confidence interval as the variance of the ration. For example, confident interval may be determined using the formula:

$$R = A / B : var ( R ) ∼ = var ( B ) / A \hat{} 2 + var ( A ) * B \hat{} 2 / A \hat{} 4 ,$$

where A and B are the weighted mean MAF at timepoint 1 and timepoint 2 respectively.

Clusters may be weighted based on the strength of evidence. For example, the max-VAF may indicate which is the primary clone, the number of non-CHIP variants may weight the cluster with the stronger signal; the driver weight may increase weight or select the cluster that contains the driver for that particular cancer type or molecular subtype. The weighting applied may be, for example, applying a greater weight to variants known to be drivers in the specific cancer type or molecular subtype. In an embodiment, weights may be based on max-VAF (either sample), number of non-CHIP variants, and/or driver weight (tumor-type-specific; defined in configuration file). In another embodiment, the weighting applied may be, for example, weighting somatic variants equally.

In an embodiment, classification as a molecular responder or a molecular non-responder may depend on the variant VAFs and variant weights. For example, if the MR score is the ratio of mean VAFs, then the higher VAF (i.e., more clonal variant) is likely to dominate. If the MR score uses variant weights, then the variant with the higher weight (e.g., driver variant) might dominate.

The resulting weighted mean of the MAF ratios as described or the ratio of the weighted means of the MAFs can be the MR score for the subject. Such an MR score incorporates the variance of MAF into the molecular response calculation. This ensures molecular response scores include accurate variance, which contributes to drawing a correct conclusion from the molecular response. The MR score may be viewed as a “numerically stable” ratio of mean MAFs, which appropriately weights changes in MAF based on the precision in the MAF, and which is not susceptible to overconfident and incorrect results when MAFs are fluctuating near the limit of detection (LOD). The MR score may be compared to a threshold to determine if the subject is responding to treatment or not responding to treatment. The threshold may be and/or include, for example, from about 25% to about 75%. In some embodiments, weighting could be either based on VAF precision (e.g. position, hotspot region, coverage depth and the like) or prior knowledge of importance of that variant to the tumor (e.g. known driver or resistance mutation, or variant of uncertain (or unknown) significance).

To provide a simple example to illustrate aspects of the problem that the MR scoring methods presented herein address, consider a subject with one variant detected, with an MAF of 0.3% at baseline (T₀), and an MAF 0.1% on treatment (T₁), and a coverage at that variant position of 3000 molecules. Using pre-existing methods, the molecular response score would be:

$$\frac{0.1 \%}{0.3 \%} = 33 \% .$$

For a cutott to define “molecular responder” vs “molecular non-responder” of 50%, this subject would be a “molecular responder.” However, propagating the variance according to the methods described herein results in a molecular response score with an expected value of ˜30-40%, but a 95% confidence interval of 0-120%. Therefore, for this subject, the molecular response should be considered not evaluable, because it cannot be confidently assessed whether the MR score is truly below or above the 50% cutoff.

To provide a simple example to illustrate aspects of the problem that the MR scoring methods presented herein address, consider a subject with two variants (a and b) detected, with MAFs of a=0.1% and b=8.0% at baseline (T₀), and MAFs a=0.3% and b=2.0% on treatment (T₁). Using pre-existing methods taking the mean of ratios, the molecular response score would be:

$$mean ( \frac{0.3 \%}{0.1 \%} , \frac{8. \%}{2. \%} ) = 163 \% .$$

For a cutoff to define “molecular responder” vs “molecular non-responder” of 50%, this subject would be a “molecular non-responder.” However, using the ratio of means according to the methods described herein the molecular response score would be

$$\frac{mean ( 0.3 \% , 2. \% )}{mean ( 0.1 \% , 8. \% )} = 28 \% .$$

Therefore, for this subject, the molecular response should be considered “molecular responder.”

To provide a simple example to illustrate aspects of the problem that the MR scoring methods presented herein address, consider a subject with two variants (a and b) detected, with MAFs of a=0.3% and b=0.0% at baseline (T₀), and MAFs a=0.0% and b=0.3% on treatment (T₁). Using pre-existing methods to only evaluate variants above 0.3% at baseline, the molecular response score would be:

$$\frac{0. \%}{0.3 \%} = 0 \% .$$

For a cutoff to define “molecular responder” vs “molecular non-responder” of 50%, this subject would be a “molecular responder.” However, including variants that arise on-treatment, the molecular response score would be

$$\frac{mean ( 0.3 \% , 0. \% )}{mean ( 0. \% , 0.3 \% )} = 100 \% .$$

Therefore, for this subject, the molecular response should be considered “molecular non-responder.”

The method 100 may include administering one or more therapies to the subject based upon at least the molecular response score. Exemplary therapies are disclosed further herein. In some embodiments, the method 100 includes comparing the molecular response score for the subject having the cancer to a predetermined cutoff point to identify that the subject is a likely responder to one or more therapies (e.g., immunotherapies or the like) for the cancer when the molecular response score is below the predetermined cutoff point or that the subject is a likely non-responder to the one or more therapies for the cancer when the molecular response score is at or above the predetermined cutoff point. In some embodiments, the method 100 includes administering one or more therapies for the cancer to the subject in view of the molecular response score. In some embodiments, the method 100 includes discontinuing administering one or more therapies for the cancer to the subject in view of the molecular response score. In some embodiments, the method 100 includes using the molecular response score as a prognostic biomarker and/or a predictive biomarker for the subject.

In other exemplary embodiments, variance is incorporated into the molecular response calculation through simulation or sampling from the variance distribution of at least one variant to calculate the molecular response variance. As further disclosed herein, some applications include weighting variants based on their importance in the tumor or likelihood of tumor vs clonal hematopoeisis. Some embodiments involve integrating multiple genomic data sources to estimate tumor fraction (instead of just relying on variant (e.g., SNV, Indel and Fusion) VAFs), coverage (e.g., copy number), off-target coverage, and/or methylation, among other genomic data sources.

In some embodiments, the methods include using one or more additional genomic data sources to determine the molecular response score for the subject having the cancer. In some embodiments, the additional genomic data sources comprise one or more of: a coverage, an off-target coverage, an epigenetic signature, tumor mutational burden and/or a microsatellite instability score. For a data source, there can be a calculation of tumor fraction based on that data source, and the calculated tumor fraction may be combined across data sources (for example using a weighted mean, incorporating the confidence of a data source in the tumor fraction for that particular sample), and then the overall tumor fraction estimate in a sample may be combined to calculate an overall molecular response. In some embodiments, the epigenetic signature comprises a cfNA fragment length, position, and/or endpoint density distribution. In some embodiments, the epigenetic signature comprises an epigenetic state or status exhibited by one or more epigenetic loci in a given targeted genomic region. In some embodiments, the epigenetic state or status comprises a presence or absence of methylation, hydroxymethylation, acctylation, ubiquitylation, phosphorylation, sumoylation, ribosylation, citrullination, and/or a histone post-translational modification or other histone variation.

While the present methods are described in the context of a first time T₀ and second time T₁, it is to be understood that more than two time points are contemplated, for example for longitudinal monitoring. At the first time T₀, baseline cfDNA may be obtained from one or more baseline samples obtained from one or more subjects prior to treatment and at a second time T₁, or any subsequent time Tn, on-treatment cfDNA may be obtained from one or more on-treatment samples obtained from one or more subjects after treatment. Time T₁ can be any amount of time after time T₀, for example, any time between and including 1-24 hours, 1-180 days, 1-12 weeks, 1-25 weeks, 1-30 weeks and the like. Moreover, the method 100 may be applied to any combinations of times T₀, T₁, . . . , Tₙ. For example, samples may be obtained at time T₁ and at a time T₂, wherein samples taken at both times are on-treatment samples. In another example, samples may be obtained at time T₁ and at a time T₂, wherein a sample taken at time T₁ represents an on-treatment sample and a sample taken at time T₂, represents an off-treatment sample.

In an embodiment, a dosage of a therapy being administered to the subject may be adjusted based on the molecular response score. For example, the molecular response score may indicate that the subject is not responding to a first treatment and the dosage of the first treatment may be increased in response. In an embodiment, an alternative therapy may be identified based on the molecular response score. For example, the molecular response score may indicate that the subject is not responding to a first treatment and the subject may then be placed on a second treatment in place of, or in addition to, the first treatment. In an embodiment, a molecular response score may be determined for subjects in a clinical trial, wherein molecular response scores may be determined for subjects receiving a placebo and for subjects receiving treatment. The molecular response scores of the two categories of subjects may be compared to assess the treatment.

In another example, placebo and treatment may be generalized to two arms of a clinical trial comparing different combinations of drugs. The threshold or cutoff may be specific to the use case: the use case may require clearance (MR=0) or the use case may require a certain level of decrease or increase of ctDNA level.

Described is an example practical application of the molecular response score for patient stratification. Advanced cancer patients may have a baseline MAF determined at time T₀, prior to treatment. After 4-10 weeks of treatment, the advanced cancer patients may have an on-treatment MAF determined at time T₁. The resulting molecular response score may indicate that ctDNA in a patient is decreasing, in which case the patient should continue to be treated with the primary trial drug. The resulting molecular response score may indicate that ctDNA in a patient is increasing, in which case the patient should continue to be treated with the primary trial drug (or with placebo) if the patient is in a control group. Otherwise, if ctDNA in a patient is increasing, the patient should have one or more therapies added to their treatment regime, a therapy changed, or a dose of the primary trial drug changed. Further details are found in PCT App. No. PCT/US2022/070984.

### III. Cancer and Other Diseases

In certain embodiments, the methods and aspects disclosed herein are used for longitudinal monitoring of patients with a given disease, disorder or condition. The methods disclosed may be used to track the response of a patient to one or more treatments over time. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

### IV. Customized Therapies and Related Administrations

In some embodiments, the methods disclosed herein relate to identifying and administering therapies to patients having a given disease, disorder or condition. Essentially any cancer therapy (e.g., surgical therapy, radiation therapy, chemotherapy, and/or the like) is included as part of these methods. In certain embodiments, the therapy administered to a subject may comprise at least one chemotherapy drug. In some embodiments, the chemotherapy drug may comprise alkylating agents (for example, but not limited to, Chlorambucil,

Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In certain embodiments, the customized therapies described herein are typically administered parenterally (e.g., intravenously or subcutaneously). Pharmaceutical compositions containing the immunotherapeutic agent are typically administered intravenously. Certain therapeutic agents are administered orally. However, customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

### V. Computer Systems, Processing of Real World Evidence (RWE)

Methods of the present disclosure can be implemented using, or with the aid of, computer systems. For example, such methods may comprise: partitioning the sample into a plurality of subsamples, including a first subsample and a second subsample, wherein the first subsample includes DNA with a cytosine modification in a greater proportion than the second subsample; subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity; and sequencing DNA in the first subsample and DNA in the second subsample in a manner that distinguishes the first nucleobase from the second nucleobase in the DNA of the first subsample.

In an aspect, the present disclosure provides a non-transitory computer-readable medium including computer-executable instructions which, when executed by at least one electronic processor, perform at least a portion of a method including: collecting cfDNA from a test subject; capturing a plurality of sets of target regions from the cfDNA, wherein the plurality of target region sets includes a sequence-variable target region set and an epigenetic target region set, whereby a captured set of cfDNA molecules is produced; sequencing the captured cfDNA molecules, wherein the captured cfDNA molecules of the sequence-variable target region set are sequenced to a greater depth of sequencing than the captured cfDNA molecules of the epigenetic target region set; obtaining a plurality of sequence reads generated by a nucleic acid sequencer from sequencing the captured cfDNA molecules; mapping the plurality of sequence reads to one or more reference sequences to generate mapped sequence reads; and processing the mapped sequence reads corresponding to the sequence-variable target region set and to the epigenetic target region set to determine the likelihood that the subject has cancer.

The code can be pre-compiled and configured for use with a machine with a processer adapted to execute the code or can be compiled during runtime. The code can be supplied in a programming language that can be selected to enable the code to execute in a pre-compiled or as-compiled fashion.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety. Further information is found in PCT Pub. No. US2022032250 and U.S. application Ser. No. 17/832,498.

Described herein is a method to generate an integrated data repository that includes multiple types of healthcare data, according to one or more implementations. The architecture may include a data integration and analysis system. The data integration and analysis system may obtain data from a number of data sources and integrate the data from the data sources into an integrated data repository. For example, the data integration and analysis system may obtain data from a health insurance claims data repository. In various examples, the data integration and analysis system and the health insurance claims data repository may be created and maintained by different entities. In one or more additional examples, the data integration and analysis system and the health insurance claims data repository may be created and maintained by the same entity.

The data integration and analysis system may be implemented by one or more computing devices. The one or more computing devices may include one or more server computing devices, one or more desktop computing devices, one or more laptop computing devices, one or more tablet computing devices, one or more mobile computing devices, or combinations thereof. In certain implementations, at least a portion of the one or more computing devices may be implemented in a distributed computing environment. For example, at least a portion of the one or more computing devices may be implemented in a cloud computing architecture. In scenarios where the computing systems used to implement the data integration and analysis system are configured in a distributed computing architecture, processing operations may be performed concurrently by multiple virtual machines. In various examples, the data integration and analysis system may implement multithreading techniques. The implementation of a distributed computing architecture and multithreading techniques cause the data integration and analysis system to utilize fewer computing resources in relation to computing architectures that do not implement these techniques.

The health insurance claims data repository may store information obtained from one or more health insurance companies that corresponds to insurance claims made by subscribers of the one or more health insurance companies. The health insurance claims data repository may be arranged (e.g., sorted) by patient identifier. The patient identifier may be based on the patient's first name, last name, date of birth, social security number, address, employer, and the like. The data stored by the health insurance claims data repository may include structured data that is arranged in one or more data tables. The one or more data tables storing the structured data may include a number of rows and a number of columns that indicate information about health insurance claims made by subscribers of one or more health insurance companies in relation to procedures and/or treatments received by the subscribers from healthcare providers. At least a portion of the rows and columns of the data tables stored by the health insurance claims data repository may include health insurance codes that may indicate diagnoses of biological conditions, and treatments and/or procedures obtained by subscribers of the one or more health insurance companies. In various examples, the health insurance codes may also indicate diagnostic procedures obtained by individuals that are related to one or more biological conditions that may be present in the individuals. In one or more examples, a diagnostic procedure may provide information used in the detection of the presence of a biological condition. A diagnostic procedure may also provide information used to determine a progression of a biological condition. In one or more illustrative examples, a diagnostic procedure may include one or more imaging procedures, one or more assays, one or more laboratory procedures, one or more combinations thereof, and the like.

The data integration and analysis system may also obtain information from a molecular data repository. The molecular data repository may store data of a number of individuals related to genomic information, genetic information, metabolomic information, transcriptomic information, fragmentomic information, immune receptor information, methylation information, epigenomic information, and/or proteomic information. In one or more examples, the data integration and analysis system and the molecular data repository may be created and maintained by different entities. In one or more additional examples, the data integration and analysis system and the molecular data repository may be created and maintained by a same entity.

The genomic information may indicate one or more mutations corresponding to genes of the individuals. A mutation to a gene of individuals may correspond to differences between a sequence of nucleic acids of the individuals and one or more reference genomes. The reference genome may include a known reference genome, such as hg19. In various examples, a mutation of a gene of an individual may correspond to a difference in a germline gene of an individual in relation to the reference genome. In one or more additional examples, the reference genome may include a germline genome of an individual. In one or more further examples, a mutation to a gene of an individual may include a somatic mutation. Mutations to genes of individuals may be related to insertions, deletions, single nucleotide variants, loss of heterozygosity, duplication, amplification, translocation, fusion genes, or one or more combinations thereof.

In one or more illustrative examples, genomic information stored by the molecular data repository may include genomic profiles of tumor cells present within individuals. In these situations, the genomic information may be derived from an analysis of genetic material, such as deoxyribonucleic acid (DNA) and/or ribonucleic acid (RNA) from a sample, including, but not limited to, a tissue sample or tumor biopsy, circulating tumor cells (CTCs), exosomes or efferosomes, or from circulating nucleic acids (e.g., cell-free DNA) found in blood samples of individuals that is present due to the degradation of tumor cells present in the individuals. In one or more examples, the genomic information of tumor cells of individuals may correspond to one or more target regions. One or more mutations present with respect to the one or more target regions may indicate the presence of tumor cells in individuals. The genomic information stored by the molecular data repository may be generated in relation to an assay or other diagnostic test that may determine one or more mutations with respect to one or more target regions of the reference genome.

In one or more additional examples, the data integration and analysis system may obtain information from one or more additional data repositories. The one or more additional data repositories may store data related to electronic medical records of individuals for which data is present in at least one of the health insurance claims data repository or the molecular data repository. Further, the one or more additional data repositories may store data related to pathology reports of individuals for which data is present in at least one of the health insurance claims data repository or the molecular data repository. In various examples, the one or more additional data repositories may store data related to biological conditions and/or treatments for biological conditions. In one or more examples, the data integration and analysis system and at least a portion of the one or more additional data repositories may be created and maintained by different entities. In one or more further examples, the data integration and analysis system and at least a portion of the one or more additional data repositories may be created and maintained by a same entity.

In one or more further implementations, the data integration and analysis system may obtain information from one or more reference information data repositories. The one or more reference information data repositories may store information that includes definitions, standards, protocols, vocabularies, one or more combinations thereof, and the like. In various examples, the information stored by the one or more reference information data repositories may correspond to biological conditions and/or treatments for biological conditions. In one or more illustrative examples, the one or more reference information data repositories may include RxNorm. (RxNorm provides normalized names for clinical drugs and links its names to many of the drug vocabularies used in pharmacy management and drug interaction software.) In one or more examples, the data integration and analysis system and at least a portion of the one or more reference information data repositories may be created and maintained by different entities. In one or more further examples, the data integration and analysis system and at least a portion of the one or more reference information data repositories may be created and maintained by a same entity.

The data integration and analysis system may obtain data from at least one of the health insurance claims data repository, the molecular data repository, the one or more additional data repositories, or the reference information data repositories via one or more communication networks accessible to the data integration and analysis system and accessible to at least one of the health insurance claims data repository, the molecular data repository, the one or more additional data repositories, or the reference information data repositories. The data integration and analysis system may also obtain data from at least one of the health insurance claims data repository, the molecular data repository, the one or more additional data repositories, or the reference information data repositories via one or more secure communication channels. In addition, the data integration and analysis system may obtain data from at least one of the health insurance claims data repository, the molecular data repository, the one or more additional data repositories, or the reference information data repositories via one or more calls of an application programming interface (API).

The data integration and analysis system may include a data integration system. The data integration system may obtain data from the health insurance claims data repository and the molecular data repository to generate the integrated data repository. The data integration system may also obtain data from the one or more additional data repositories to generate the integrated data repository. In various examples, the data integration system may implement one or more natural language processing techniques to integrate data from the one or more additional data repositories into the integrated data repository.

In one or more examples, the data integration system may generate one or more tokens to identify individuals that have data stored in the health insurance claims data repository and that have data stored in the molecular data repository. In various examples, the data integration system may generate one or more tokens by implementing one or more hash functions. The data integration system may implement the one or more hash functions to generate the one or more tokens based on information stored by at least one of the health insurance claims data repository or the molecular data repository. For example, the information used by the data integration system to generate individual tokens by implementing a hash function may include at least one of an identifiers of respective individuals, date of birth of the respective individuals, a postal code of the respective individuals, date of birth of the respective individuals, or a gender of the respective individuals. In one or more illustrative examples, the identifiers of the respective individuals may include a combination of at least a portion of a first name of the respective individuals and at least a portion of the last name of the respective individuals. Tokens generated using data from different data repositories may correspond to the same or similar information or the same or similar type stored by the different data repositories. To illustrate, tokens may be generated using a portion of names of individuals, date of birth, at least a portion of a postal code, and gender obtained from the health insurance claims data repository and the molecular data repository.

The data integration system may integrate data from a number of different data sources by analyzing tokens generated by implementing one or more hash functions using data obtained from the number of different data sources. For example, the data integration system may obtain one or more first tokens generated from data stored by the health insurance claims data repository and one or more second tokens generated from data stored by the molecular data repository. The data integration system may analyze the one or more first tokens with respect to the one or more second tokens to determine individual first tokens that correspond to individual second tokens. In one or more illustrative examples, the data integration system may identify individual first tokens that match individual second tokens. A first token may match a second token when the data of the first token has at least a threshold amount of similarity with respect to the data of the second token. In one or more examples, a first token may match a second token when the data of the first token is the same as the data of the second token. To illustrate, a first token may match a second token when an alphanumeric string of the first token is the same as an alphanumeric string of the second token.

By determining a first token generated using data stored by the health insurance claims data repository that corresponds to a second token generated using data stored by the molecular data repository, the data integration system may identify an individual having data that is stored in both the health insurance claims data repository and in the molecular data repository. In this way, the data integration system may obtain data from the health insurance claims data repository from a number of individuals and data from the molecular data repository from the same number of individuals and store the health insurance claims data and the molecular data for the number of individuals in the integrated data repository.

The data integration system may also integrate data stored by the one or more additional data repositories with data from the health insurance claims data repository and the molecular data repository to generate the integrated data repository. To illustrate, the data integration system may obtain one or more thirds of tokens generated from data stored by an additional data repository, such as a data repository storing data corresponding to pathology reports. The data integration system may analyze the one or more third tokens with respect to the first tokens generated using information stored by the health insurance claims data repository and the second tokens generated using information stored by the molecular data repository to determine respective third tokens that correspond to individuals first tokens and individual second tokens. In one or more illustrative examples, the data integration system may identify third tokens generated using one or more hash functions and a common set of information obtained from the health insurance claims data repository, the molecular data repository, and the additional data repository.

By determining a third token generated using data stored by an additional data repository that corresponds to a first token generated using data stored by the health insurance claims data repository and a second token generated using data stored by the molecular data repository, the data integration system may identify an individual having data that is stored in the health insurance claims data repository, the molecular data repository, and in an additional data repository. In this way, the data integration system may obtain data from the health insurance claims data repository from a number of individuals and data from the molecular data repository and an additional data repository from the same number of individuals and store the health insurance claims data, the molecular data, and the additional data for the number of individuals in the integrated data repository.

The data stored by the integrated data repository for the number of individuals may be accessible using respective identifiers of individuals. The data integration system may implement a number of techniques as part of a de-identification process with respect to storing and retrieving information of individuals in the integrated data repository. The identifiers of individuals may correspond to keys that are generated using at least one hash function. The identifiers of the individuals may also be generated by implementing one or more salting processes with respect to the keys generated using the at least one hash function, the tokens generated using one or more hash functions and a common set of information obtained from the health insurance claims data repository, the molecular data repository, and/or the additional data repository. In one or more illustrative examples, the identifiers generated by the data integration system to access information for respective individuals that is stored by the integrated data repository may be unique for each individual. In one or more examples, the identifiers of the individuals may be generated using at least a portion of the information used to generate the tokens related to the individuals. In one or more additional examples, the identifiers of the individuals may be generated using different information from the information used to generate the tokens related to the individuals.

The data integration system may also generate the integrated data repository from a number of different combinations of data repositories in a similar manner. For example, the data integration system may obtain tokens generated from information stored by the health insurance claims data repository and additional tokens generated from information stored by one or more additional data stores. The data integration system may determine individual tokens generated from information stored by the health insurance claims data repository that correspond to individual additional tokens generated from information stored by the one or more additional data repositories. By determining tokens generated using data stored by the health insurance claims data repository that correspond to additional tokens generated using data stored by an additional data repository, the data integration system may identify individuals having data that is stored in both the health insurance claims data repository and in the additional data repository. In this way, the data integration system may obtain data from the health insurance claims data repository from a number of individuals and data from the additional data repository from the same number of individuals and store the health insurance claims data and the additional data for the number of individuals in the integrated data repository. The health insurance claims data and the additional data stored by the integrated data repository for the number of individuals may be accessible using respective identifiers of individuals.

In one or more further examples, the data integration system may obtain tokens generated from information stored by the molecular data repository and tokens generated from information stored by one or more additional data stores. The data integration system may determine individual tokens generated from information stored by the molecular data repository that correspond to individual additional tokens generated from information stored by the one or more additional data repositories. By determining tokens generated using data stored by the molecular data repository that correspond to additional tokens generated using data stored by an additional data repository, the data integration system may identify individuals having data that is stored in both the molecular data repository and in the additional data repository. In this way, the data integration system may obtain data from the molecular data repository from a number of individuals and data from the additional data repository from the same number of individuals and store the molecular data and the additional data for the number of individuals in the integrated data repository. The molecular data and the additional data stored by the integrated data repository for the number of individuals may be accessible using respective identifiers of individuals.

The data stored by the integrated data repository may be stored according to one or more regulatory frameworks that protect the privacy and ensure the security of medical records, health information, and insurance information of individuals. For example, data may be stored by the integrated data repository in accordance with one or more governmental regulatory frameworks directed to protecting personal information, such as the Health Insurance Portability and Accountability Act (HIPAA) and/or the General Data Protection Regulation (GDPR). The integrated data repository also stores data in an anonymized and de-identified manner to ensure protection of the privacy of individuals that have data stored by the integrated data repository. To further ensure the privacy of individuals that have data stored by the integrated data repository, the data integration system may re-generate the integrated data repository periodically. For example, the data integration system may create the integrated data repository once per quarter. In one or more additional examples, the data integration system may generate the integrated data repository on a monthly basis, on a weekly basis, or once every two weeks. By re-generating the integrated data repository on a periodic basis and not simply refreshing the integrated data repository when new data is available, the integrated data repository enhances privacy protection with respect to data stored by the integrated data repository. That is, in situations where data repositories are refreshed simply with new data, it may be possible to more easily track individuals associated with data that has been newly added to a data repository because the number of new individuals added at a given time is typically smaller than an existing number of individuals that already have data stored by the data repository.

In various examples, data stored by the integrated data repository may be accessed via a database management system. In addition, the integrated data repository may store data according to one or more database models. In one or more examples, the integrated data repository may store data according to one or more relational database technologies. For example, the integrated data repository may store data according to a relational database model. In one or more additional examples, the integrated data repository may store data according to an object-oriented database model. In one or more further examples, the integrated data repository may store data according to an extensible markup language (XML) database model. In additional examples, the integrated data repository may store data according to a structured query language (SQL) database model. In still further examples, the integrated data repository may store data according to an image database model.

The data integration system may generate the integrated data repository by generating a number of data tables and creating links between the data tables. The links may indicate logical couplings between the data tables. The data integration system may generate the data tables by extracting specified sets of data from the information obtained from the data repositories and storing the data in rows and columns of respective data tables. In various examples, the logical couplings between data tables may include at least one of a one-to-one link where a row of information in one data table corresponds to a row of information in another data table, a one-to-many link where a row of information in one data table corresponds to multiple rows of information in another data table, or a many-to-many link where multiple rows of information of one data table correspond to multiple rows of information in another data table.

The number of data tables may be arranged according to a data repository schema. In the illustrative example of the data repository schema includes a first data table, a second data table, a third data table, a fourth data table, and a fifth data table. Although the illustrative example of includes five data tables, in additional implementations, the data repository schema may include more data tables or fewer data tables. The data repository schema may also include links between the data tables. The links between the data tables may indicate that information retrieved from one of the data tables results in additional information stored by one or more additional data tables to be retrieved. Additionally, not all the data tables may be linked to each of the other data tables. In the illustrative example of the first data table is logically coupled to the second data table by a first link and the first data table is logically coupled to the fourth data table by a second link. In addition, the second data table is logically coupled to the third data table via a third link and the fourth data table is logically coupled to the fifth data table via a fourth link. Further, the third data table is logically coupled to the fifth data table via a fifth link.

In various examples, as data tables are added to and/or removed from the data repository schema, additional links between data tables may be added to or removed from the data repository schema. In one or more illustrative examples, the integrated data repository may store data tables according to the data repository schema for at least a portion of the individuals for which the data integration system obtained information from a combination of at least two of the health insurance claims data repository, the molecular data repository, the one or more additional data repositories, and the one or more reference information data repositories. As a result, the integrated data repository may store respective instances of the data tables according to the data repository schema for thousands, tens of thousands, up to hundreds of thousands or more individuals.

The data integration and analysis system may also include a data pipeline system. The data pipeline system may include a number of algorithms, software code, scripts, macros, or other bundles of computer-executable instructions that process information stored by the integrated data repository to generate additional datasets. The additional datasets may include information obtained from one or more of the data tables. The additional datasets may also include information that is derived from data obtained from one or more of the data tables. The components of the data pipeline system implemented to generate a first additional dataset may be different from the components of the data pipeline system used to generate a second additional dataset.

In one or more examples, the data pipeline system may generate a dataset that indicates pharmacy treatments received by a number of individuals. In one or more illustrative examples, the data pipeline system may analyze information stored in at least one of the data tables to determine health insurance codes corresponding to pharmaceutical treatments received by a number of individuals. The data pipeline system may analyze the health insurance codes corresponding to pharmaceutical treatments with respect to a library of data that indicates specified pharmaceutical treatments that correspond to one or more health insurance codes to determine names of pharmaceutical treatments that have been received by the individuals. In one or more additional examples, the data pipeline system may analyze information stored by the integrated data repository to determine medical procedures received by a number of individuals. To illustrate, the data pipeline system may analyze information stored by one of the data tables to determine treatments received by individuals via at least one injection or intravenously. In one or more further examples, the data pipeline system may analyze information stored by the integrated data repository to determine episodes of care for individuals, lines of therapy received by individuals, progression of a biological condition, or time to next treatment. In various examples, the datasets generated by the data pipeline system may be different for different biological conditions. For example, the data pipeline system may generate a first number of datasets with respect to a first type of cancer, such as lung cancer, and a second number of datasets with respect to a second type of cancer, such as colorectal cancer.

The data pipeline system may also determine one or more confidence levels to assign to information associated with individuals having data stored by the integrated data repository. The respective confidence levels may correspond to different measures of accuracy for information associated with individuals having data stored by the integrated data repository. The information associated with the respective confidence levels may correspond to one or more characteristics of individuals derived from data stored by the integrated data repository. Values of confidence levels for the one or more characteristics may be generated by the data pipeline system in conjunction with generating one or more datasets from the integrated data repository. In one or more examples, a first confidence level may correspond to a first range of measures of accuracy, a second confidence level may correspond to a second range of measures of accuracy, and a third confidence level may correspond to a third range of measures of accuracy. In one or more additional examples, the second range of measures of accuracy may include values that are less values of the first range of measures of accuracy and the third range of measures of accuracy may include values that are less than values of the second range of measures of accuracy. In one or more illustrative examples, information corresponding to the first confidence level may be referred to as Gold standard information, information corresponding to the second confidence level may be referred to as Silver standard information, and information corresponding to the third confidence level may be referred to as Bronze standard information. The data pipeline system may determine values for the confidence levels of characteristics of individuals based on a number of factors. For example, a respective set of information may be used to determine characteristics of individuals. The data pipeline system may determine the confidence levels of characteristics of individuals based on an amount of completeness of the respective set of information used to determine a characteristic for an individual. In situations where one or more pieces of information are missing from the set of information associated with a first number of individuals, the confidence levels for a characteristic may be lower than for a second number of individuals where information is not missing from the set of information. In one or more examples, an amount of missing information may be used by the data pipeline system to determine confidence levels of characteristics of individuals. To illustrate, a greater amount of missing information used to determine a characteristic of an individual may cause confidence levels for the characteristic to be lower than in situations where the amount of missing information used to determine the characteristic is lower. Further, different types of information may correspond to various confidence levels for a characteristic. In one or more examples, the presence of a first piece of information used to determine a characteristic of an individual may result in confidence levels for the characteristic being higher than the presence of a second piece of information used to determine the characteristic.

In one or more illustrative examples, the data pipeline system may determine a number of individuals included in a cohort with a primary diagnosis of lung cancer (or other biological condition). The data pipeline system may determine confidence levels for respective individuals with respect to being classified as having a primary diagnosis of lung cancer. The data pipeline system may use information from a number of columns included in the data tables to determine a confidence level for the inclusion of individuals within a lung cancer cohort. The number of columns may include health insurance codes related to diagnosis of biological conditions and/or treatments of biological conditions. Additionally, the number of columns may correspond to dates of diagnosis and/or treatment for biological conditions. The data pipeline system may determine that a confidence level of an individual being characterized as being part of the lung cancer cohort is higher in scenarios where information is available for each of the number of columns or at least a threshold number of columns than in instances where information is available for less than a threshold number of columns. Further, the data pipeline system may determine confidence levels for individuals included in a lung cancer cohort based on the type of information and availability of information associated with one or more columns. To illustrate, in situations where one or more diagnosis codes are present in relation to one or more periods of time for a group of individuals and one or more treatment codes are absent, the data pipeline system may determine that the confidence level of including the group of individuals in the lung cancer cohort is greater than in situations where at least one of the diagnosis codes is absent and the treatment codes used to determine whether individuals are included in the lung cancer cohort are present.

The data integration and analysis system may include a data analysis system. The data analysis system may receive integrated data repository requests from one or more computing devices, such as an example computing device. The one or more integrated data repository requests may cause data to be retrieved from the integrated data repository. In various examples, the one or more integrated data repository requests may cause data to be retrieved from one or more datasets generated by the data pipeline system. The integrated data repository requests may specify the data to be retrieved from the integrated data repository and/or the one or more datasets generated by the data pipeline system. In one or more additional examples, the integrated data repository requests may include one or more prebuilt queries that correspond to computer-executable instructions that retrieve a specified set of data from the integrated data repository and/or one or more datasets generated by the data pipeline system.

In response to one or more integrated data repository requests, the data analysis system may analyze data retrieved from at least one of the integrated data repository or one or more datasets generated by the data pipeline system to generate data analysis results. The data analysis results may be sent to one or more computing devices, such as example computing devices. Although the illustrative example of hows that the one or more integrated data repository requests from one computing device and the data analysis results being sent to another computing device, in one or more additional implementations, the data analysis results may be received by a same computing device that sent the one or more integrated data repository requests. The data analysis results may be displayed by one or more user interfaces rendered by the computing device or the computing device.

In one or more examples, the data analysis system may implement at least one of one or more machine learning techniques or one or more statistical techniques to analyze data retrieved in response to one or more integrated data repository requests. In one or more examples, the data analysis system may implement one or more artificial neural networks to analyze data retrieved in response to one or more integrated data repository requests. To illustrate, the data analysis system may implement at least one of one or more convolutional neural networks or one or more residual neural networks to analyze data retrieved from the integrated data repository in response to one or more integrated data repository requests. In at least some examples, the data analysis system may implement one or more random forests techniques, one or more support vector machines, or one or more Hidden Markov models to analyze data retrieved in response to one or more integrated data repository requests. One or more statistical models may also be implemented to analyze data retrieved in response to one or more integrated data repository requests to identify at least one of correlations or measures of significance between characteristics of individuals. For example, log rank tests may be applied to data retrieved in response to one or more integrated data repository requests. In addition, Cox proportional hazards models may be implemented with respect to date retrieved in response to one or more integrated data repository requests. Further, Wilcoxon signed rank tests may be applied to data retrieved in response to one or more integrated data repository requests. In still other examples, a z-score analysis may be performed with respect to data retrieved in response to one or more integrated data repository requests. In still additional examples, a Kaplan Meier analysis may be performed with respect to data retrieved in response to one or more integrated data repository requests. In at least some examples, one or more machine learning techniques may be implemented in combination with one or more statistical techniques to analyze data retrieved in response to one or more integrated data repository requests.

In one or more illustrative examples, the data analysis system may determine a rate of survival of individuals in which lung cancer is present in response to one or more treatments. In one or more additional illustrative examples, the data analysis system may determine a rate of survival of individuals having one or more genomic region mutations in which lung cancer is present in response to one or more treatments. In various examples, the data analysis system may generate the data analysis results in situations where the data retrieved from at least one of the integrated data repository or the one or more datasets generated by the data pipeline system satisfies one or more criteria. For example, the data analysis system may determine whether at least a portion of the data retrieved in response to one or more integrated data repository requests satisfies a threshold confidence level. In situations where the confidence level for at least a portion of the date retrieved in response to one or more integrated data repository requests is less than a threshold confidence level, the data analysis system may refrain from generating at least a portion of data analysis results. In scenarios where the confidence level for at least a portion of the data retrieved in response to one or more integrated data repository requests is at least a threshold confidence level, the data analysis system may generate at least a portion of the data analysis results. In various examples, the threshold confidence level may be related to the type of data analysis results being generated by the data analysis system.

In one or more illustrative examples, the data analysis system may receive an integrated data repository request to generate data analysis results that indicate a rate of survival of one or more individuals. In these instances, the data analysis system may determine whether the data stored by the integrated data repository and/or by one or more datasets generated by the data pipeline system satisfies a threshold confidence level, such as a Gold standard confidence level. In one or more additional examples, the data analysis system may receive an integrated data repository request to generate data analysis results that indicate a treatment received by one or more individuals. In these implementations, the data analysis system may determine whether the data stored by the integrated data repository and/or by one or more datasets generated by the data pipeline system satisfies a lower threshold confidence level, such as a Bronze standard confidence level.

In one or more additional illustrative examples, the data analysis system may receive an integrated data repository request to determine individuals having one or more genomic mutations and that have received one or more treatments for a biological condition. Continuing with this example, the data analysis system can determine a survival rate of individuals with the one or more genomic mutations in relation to the one or more treatments received by the individuals. The data analysis system can then identify based on the survival rate of individuals and effectiveness of treatments for the individuals in relation to genomic mutations that may be present in the individuals. In this way, health outcomes of individuals may be improved by identifying prospective treatments that may be more effective for populations of individuals having one or more genomic mutations than current treatments being provided to the individuals.

Described herein is a framework corresponding to an arrangement of data tables in an integrated data repository, according to one or more implementations. In the illustrative example of the framework includes a data repository schema that includes a first data table, a second data table, a third data table, a fourth data table a fifth data table, a sixth data table, and a seventh data table. Although the illustrative example of seven data tables, in additional implementations, the data repository schema may include more data tables or fewer data tables. The data repository schema may also include links between the data tables. The links between the data tables may indicate that information retrieved from one of the data tables results in additional information stored by one or more additional data tables to be retrieved. Additionally, not all the data tables may be linked to each of the other data tables. In the illustrative example of the first data table is logically coupled to the second data table by a first link and the third data table is logically coupled to the second data table by a second link. The second data table is also logically coupled to the fourth data table by a third link, the second data table is logically coupled to the fifth data table by a fourth link, and the second data table is logically coupled to the sixth data table by a fifth link. In addition, the fifth data table is logically coupled to the sixth data table by a sixth link and the sixth data table is logically coupled to the seventh data table by a seventh link. Further, the seventh data table is logically coupled to the fourth data table by an eighth link. In various examples, as data tables are added to and/or removed from the data repository schema, additional links between data tables may be added to or removed from the data repository schema. In one or more illustrative examples, the integrated data repository may store data tables according to the data repository schema for at least a portion of the individuals for which the data integration system obtained information from a combination of at least two of the health insurance claims data repository, the molecular data repository, and the one or more additional data repositories. As a result, the integrated data repository may store respective instances of the data tables according to the data repository schema for thousands, tens of thousands, up to hundreds of thousands or more individuals.

In one or more examples, the first data table may store data corresponding to genomics and genomics testing for individuals. For example, the first data table may include columns that include information corresponding to a panel used to generate genomics data, mutations of genomic regions, types of mutations, copy numbers of genomic regions, coverage data indicating numbers of nucleic acid molecules identified in a sample having one or more mutations, testing dates, and patient information. The first data table may also include one or more columns that include health insurance data codes that may correspond to one or more diagnosis codes. Additionally, the information in the first data table may include at least one identifier for an individual that is associated with an instance of the first data table.

The second data table may store data related to one or more patient visits by individuals to one or more healthcare providers. The third data table may store information corresponding to respective services provided to individuals with respect to one or more patient visits to one or more healthcare providers indicated by the second data table. To illustrate, an individual may visit a healthcare provider and multiple services may be performed with respect to the individual at the visit. A second data table may include columns indicating information for each of the multiple services performed during the patient visit. Multiple third data tables may be generated with respect to the patient visit that include columns indicating information on a more granular level for a respective service provided during the patient visit than the information stored by the second data table related to the patient visit. For example, the second data table may include multiple columns indicating a health insurance code for different services provided to an individual during a patient visit and a third data table related to one of the services may include multiple columns for additional health insurance codes that correspond to additional information related to the respective services. The second data table and the third data table(s) for a patient visit may indicate one or more dates of service corresponding to the patient visit.

The fourth data table may include columns that indicate information about individuals for which information is stored by the integrated data repository. For example, the fourth data table may include columns that indicate information related to at least one of a location of an individual, a gender of an individual, a date of birth of an individual, a date of death of an individual (if applicable), or one or more keys associated with the individual. In one or more examples, the fourth data table may include one or more columns related to whether erroneous data has been identified for an individual. In various examples, a single fourth data table may be generated for respective individuals. Thus, the data repository schema may include multiple instances of the fourth data table, such as thousands, tens of thousands, up to hundreds of thousands or more.

The fifth data table may include columns that indicate information related to a health insurance company or governmental entity that made payment for one or more services provided to respective individuals. For example, the fifth data table may include one or more payer identifiers. The sixth data table may include columns that include information corresponding to health insurance coverage information for respective individuals. In one or more examples, the sixth data table may include columns indicating the presence of medical coverage for an individual, the presence of pharmacy coverage for an individual, and a type of health insurance plan related to the individual, such as health maintenance organization (HMO), preferred provider organization (PPO), and the like.

The seventh data table may include columns that indicate information related to pharmaceutical treatments obtained by a respective individual. In one or more examples, the seventh data table may include one or more columns indicating health insurance codes corresponding to pharmaceutical treatments that are available via a pharmacy. The health insurance codes may correspond to individual pharmaceutical treatments. Additionally, the health insurance codes may indicate a diagnosis of a biological condition with respect to an individual. The seventh data table may also include additional information, such as at least one of dosage amounts, number of days' supply, quantity dispensed, number of refills authorized, dates of service, or information related to the individual receiving the pharmaceutical treatment.

In various examples, the data repository schema may provide results of analysis of the information stored by the data tables in a more efficient manner than typical data repository schemas. For example, the logical connections between the data tables are arranged to efficiently retrieve data that is related across the different data tables. In situations where the data tables are arranged in a serial manner and/or in situations where a greater number of the data tables are logically connected, retrieving data from the integrated data repository from one or more of the data tables to responds to a request for information from the integrated data repository will be less efficient than in situations where the data repository schema is implemented.

Described herein is an architecture to generate one or more datasets from information retrieved from a data repository that integrates health related data from a number of sources, according to one or more implementations. The architecture may include the data integration and analysis system and the integrated data repository. Additionally, the data integration and analysis system may include at least the data pipeline system and the data analysis system. The data pipeline system may include a number of sets of data processing instructions that are executable to generate respective datasets that may be analyzed by the data analysis system in response to an integrated data repository request to generate data analysis results.

The data pipeline system may include first data processing instructions, second data processing instructions, up to Nth data processing instructions. The data processing instructions, may be executable by one or more processing units to perform a number of operations to generate respective datasets using information obtained from the integrated data repository. In one or more illustrative examples, the data processing instructions, may include at least one of software code, scripts, API calls, macros, and so forth. The first data processing instructions may be executable to generate a first dataset. In addition, the second data processing instructions may be executable to generate a second dataset. Further, the Nth data processing instructions may be executable to generate an Nth dataset. In various examples, after the data integration and analysis system generates the integrated data repository, the data pipeline system may cause the data processing instructions, to be executed to generate the datasets. In one or more examples, the datasets, may be stored by the integrated data repository or by an additional data repository that is accessible to the data integration and analysis system. At least a portion of the data processing instructions may analyze health insurance codes to generate at least a portion of the datasets. Additionally, at least a portion of the data processing instructions may analyze genomics data to generate at least a portion of the datasets.

In one or more examples, the first data processing instructions may be executable to retrieve data from one or more first data tables stored by the integrated data repository. The first data processing instructions may also be executable to retrieve data from one or more specified columns of the one or more first data tables. In various examples, the first data processing instructions may be executable to identify individuals that have a health insurance code stored in one or more column and row combinations that correspond to one or more diagnosis codes. The first data processing instructions may then be executable to analyze the one or more diagnosis codes to determine a biological condition for which the individuals have been diagnosed. In one or more illustrative examples, the first data processing instructions may be executable to analyze the one or more diagnosis codes with respect to a library of diagnosis codes that indicates one or more biological conditions that correspond to respective diagnosis codes. The library of diagnosis codes may include hundreds up to thousands of diagnosis codes. The first data processing instructions may also be executable to determine individuals diagnosed with a biological condition by analyzing timing information of the individuals, such as dates of treatment, dates of diagnosis, dates of death, one or more combinations thereof, and the like.

The second data processing instructions may be executable to retrieve data from one or more second data tables stored by the integrated data repository. The second data processing instructions may also be executable to retrieve data from one or more specified columns of the one or more second data tables. In various examples, the second data processing instructions may be executable to identify individuals that have a health insurance code stored in one or more columns and row combinations that correspond to one or more treatment codes. The one or more treatment codes may correspond to treatments obtained from a pharmacy. In one or more additional examples, the one or more treatment codes may correspond to treatments received by a medical procedure, such as an injection or intravenously. The second data processing instructions may be executable to determine one or more treatments that correspond to the respective health insurance codes included in the one or more second data tables by analyzing the health insurance code in relation to a predetermined set of information. The predetermined set of information may include a data library that indicates one or more treatments that correspond to one out of hundreds up to thousands of health insurance codes. The second data processing instructions may generate the second dataset to indicate respective treatments received by a group of individuals. In one or more illustrative examples, the group of individuals may correspond to the individuals included in the first dataset. The second dataset may be arranged in rows and columns with one or more rows corresponding to a single individual and one or more columns indicating the treatments received by the respective individual.

The Nth processing instructions (where N may be any positive integer) may be executable to generate the Nth dataset by combining information from a number of previously generated datasets, such as the first dataset and the second dataset. In addition, the Nth processing instructions may be executable to generate the Nth dataset to retrieve additional information from one or more additional columns of the integrated data repository and incorporate the additional information from the integrated data repository with information obtained from the first dataset and the second dataset. For example, the Nth processing instructions may be executable to identify individuals included in the first dataset that are diagnosed with a biological condition and analyze specified columns of one or more additional data tables of the integrated data repository to determine dates of the treatments indicated in the second dataset that correspond to the individuals included in the first dataset. In one or more further examples, the Nth processing instructions may be executable to analyze columns of one or more additional data tables of the integrated data repository to determine dosages of treatments indicated in the second dataset received by the individuals included in the first dataset. In this way, the Nth processing instructions may be executable to generate an episodes of care dataset based on information included in a cohort dataset and a treatments dataset.

In one or more illustrative examples, in response to receiving an integrated data repository request, the data analysis system may determine one or more datasets that correspond to the features of the query related to the integrated data repository request. For example, the data analysis system may determine that information included in the first dataset and the second dataset is applicable to responding to the integrated data repository request. In these scenarios, the data analysis system may analyze at least a portion of the data included in the first dataset and the second dataset to generate the data analysis results. In one or more additional examples, the data analysis system may determine different datasets to respond to different queries included in the integrated data repository request in order to generate the data analysis results.

The use of specific sets of data processing instructions to generate respective data sets may reduce the number of inputs from users of the data integration and analysis system as well as reduce the computational load, such as the amount of processing resources and memory, utilized to process integrated data repository requests. For example, without the specific architecture of the data pipeline system, each time an integrated data repository request is received, the data utilized to respond to the integrated data repository request is assembled from the data repository. In contrast, by implementing the data pipeline system to execute the data processing instruction to generate the datasets the data needed to respond to various integrated data repository requests has already been assembled and may be accessed by the data analysis system to respond to the integrated data repository request. Thus, the computing resources used to respond to the integrated data repository request by implementing the data pipeline system to generate the datasets are less than typical systems that perform an information parsing and collecting process for each integrated data repository request. Further, in situations where the data pipeline system has not been implemented, users of the data integration and analysis system may need to submit multiple integrated data repository request in order to analyze the information that the users are intending to have analyzed either because the ad hoc collection of data to respond to an integrated data repository request in typical systems is inaccurate or because the data analysis system is called upon multiple times to perform an analysis of information in typical systems that may be performed using a single integrated data repository request when the data pipeline system is implemented.

Described herein is an architecture to generate an integrated data repository that includes de-identified health insurance claims data and de-identified genomics data it, according to one or more implementations. The architecture may include the data integration and analysis system, the health insurance claims data repository, and the molecular data repository. The data integration and analysis system may obtain patient information from the molecular data repository. The patient information may include genomics data for individuals having data stored by the molecular data repository. The genomics data may indicate results of one or more nucleic acid sequencing operations that analyze sequences of nucleic acid molecules included in a sample obtained from the individuals with respect to one or more target genomic regions. In one or more examples, the sample may be obtained from tissue of one or more individuals. In one or more additional examples, the sample may be obtained from fluid of one or more individuals, such as blood or plasma. The one or more target genomic regions may correspond to genomic regions that correspond to the presence of one or more biological conditions. For example, the target regions may correspond to genomic regions of a reference genome having mutations that are present in individuals in which a biological condition is present. In one or more illustrative examples, the target regions may correspond to genomic regions of a reference human genome in which one or more mutations are present in individuals in which one or more forms of cancer are present. The patient information may also include information indicating personal information about individuals with data stored by the molecular data repository and information corresponding to the testing and analysis performed on samples provided by individuals.

The data integration and analysis system may perform a de-identification process that anonymizes personal information obtained from the molecular data repository. The data integration and analysis system may implement one or more computational techniques as part of the de-identification process to anonymize data related to individuals stored by the molecular data repository such that the de-identified data protects the privacy of the individuals and is in compliance with one or more privacy regulation frameworks. The de-identification process may include, at, accessing tokens. In various examples, the tokens may comprise an alphanumeric string of characters. In one or more examples, the tokens may be generated by the data integration and analysis system. In one or more additional examples the tokens may be generated by a third-party and obtained by the data integration and analysis system.

The tokens may be generated using one or more hash functions in relation to a subset of the patient information. To illustrate, for individuals that have information stored by the molecular data repository, the tokens may be generated using a combination of at least a portion of a first name of the respective individuals, at least a portion of the last name of the respective individuals, at least a portion of a date of birth of the respective individuals, a gender of the individuals, and at least a portion of a location identifier of the respective individuals. The de-identification process may also include, at, generating identifiers for individuals that have data stored by the molecular data repository. The identifiers may be generated by the data integration and analysis system using one or more hash functions that are different from the one or more hash functions used to generate the tokens. In one or more illustrative examples, the data integration and analysis system may generate an intermediate version of respective identifiers using one or more hash function and then apply one or more salting techniques to the intermediate versions of the identifiers to generate final versions of the identifiers. A salt function includes a function configured to add at least one random bit to each intermediate identifier to generate a respective final identifier. In various examples, the data integration and analysis system may generate the identifiers at using at least a portion of the information for respective individuals stored by the molecular data repository. In one or more illustrative examples, the identifiers may be generated based on a patient identifier included in the patient information. The identifiers generated by the data integration and analysis system may be unique for respective individuals having data stored by the molecular data repository.

At operation, the data integration and analysis system may generate modified patient information based on the identifiers. The modified patient information may include genomics data related to individuals associated with the molecular data repository and the identifiers of the respective individuals. The modified patient information may have a data structure. The data structure may include a column that includes respective identifiers of individuals associated with the molecular data repository and a number of columns that include genomics data related to the individuals, such as identifiers of one or more genes, alterations to the one or more genes, type of alteration to the genes, and so forth.

The data integration and analysis system may generate a token file. The token file may include first tokens accessed at operation for respective individuals having data stored by the molecular data repository. The token file may have a data structure that includes a number of columns that include information for respective individuals. The data structure may include a column indicating respective identifiers generated by the data integration and analysis system and columns indicating one or more first tokens associated with the respective identifiers. The data integration and analysis system may send the token file to a health insurance claims data management system that is coupled to the health insurance claims data repository. The health insurance claims data management system may analyze the first tokens with respect to corresponding second tokens. The second tokens may be accessed by or generated by the health insurance claims data management system. The second tokens may be generated using a same or similar subset of information for individuals having data stored in the health insurance claims data repository as the subset of the patient information. For example, the second tokens may be generated using a combination of at least a portion of a first name of the respective individuals, at least a portion of the last name of the respective individuals, at least a portion of a date of birth of the respective individuals, a gender of the individuals, and at least a portion of a location identifier of the respective individuals.

In various examples, the health insurance claims data management system may retrieve health insurance claims data from the health insurance claims data repository for individuals associated with respective second tokens that match corresponding first tokens. A first token may match a second token when the data of the first token has at least a threshold amount of similarity with respect to the data of the second token. In one or more examples, a first token may match a second token when the data of the first token is the same as the data of the second token.

In response to identifying health insurance claims data for individuals having respective second tokens that correspond to a respective first token, the health insurance claims data management system may generate modified health insurance claims data. The health insurance claims data management system may send the modified health insurance claims data to the data integration and analysis system. In one or more examples, the modified health insurance claims data may be formatted according to a data structure. The data structure may include a column that includes a subset of the second tokens that correspond to the first tokens and a number of columns that include the health insurance claims data.

At operation, the data integration and analysis system may integrate genomics data and health insurance claims data of individuals that are common to both the molecular data repository and the health insurance claims data repository. The data integration and analysis system may determine individuals that are common to both the molecular data repository and the health insurance claims data repository by determining genomics data and health insurance claims data corresponding to common tokens. The data integration and analysis system may determine that a first token related to a portion of the genomics data corresponds to a second token related to a portion of the health insurance claims data by determining a measure of similarity between the first token and the second token. In scenarios where the first token has at least a threshold amount of similarity with respect to the second token, the data integration and analysis system may store the corresponding portion of the genomics data and the corresponding portion of the health insurance claims data in relation to the identifier of the individual in an integrated data repository, such as an integrated data repository.

The implementation of the architecture may implement a cryptographic protocol that enables de-identified information from disparate data repositories to be integrated into a single data repository. In this way, the security of the data stored by the integrated data repository is increased. Additionally, the cryptographic protocol implemented by the architecture may enable more efficient retrieval and accurate analysis of information stored by the integrated data repository than in situations where the cryptographic protocol of the architecture is not utilized. For example, by generating a token file that includes first tokens using a cryptographic technique based on a specified set of information stored by the molecular data repository and utilizing second tokens generated using a same or similar cryptographic technique with respect to the similar or same set of information stored by the health insurance claims data repository, the data integration and analysis system may match information stored by disparate data repositories that correspond to a same individual. Without implementing the cryptographic protocol of the architecture, the probability of incorrectly attributing information from one data repository to one or more individuals increases, which decreases the accuracy of results provided by the data integration and analysis system in response to integrated data repository requests sent to the data integration and analysis system.

Described herein is a framework to generate a dataset, by a data pipeline system, based on data stored by an integrated data repository, according to one or more implementations. The integrated data repository may store health insurance claims data and genomics data for a group of individuals. For example, the integrated data repository may store information obtained from health insurance claims records of the group of individuals. For each individual included in the group of individuals, the integrated data repository may store information obtained from multiple health insurance claim records. In various examples, the information stored by the integrated data repository may include and/or be derived from thousands, tens of thousands, hundreds of thousands, up to millions of health insurance claims records for a number of individuals. Additionally, each health insurance claim record may include multiple columns. As a result, the integrated data repository may be generated through the analysis of millions of columns of health insurance claims data.

Further, although the health insurance claims data may be organized according to a structured data format, health insurance claims data is typically arranged to be viewed by health insurance providers, patients, and healthcare providers in order to show financial information and insurance code information related to services provided to individuals by healthcare providers. Thus, health insurance claims data is not easily analyzed to gain insights that may be available in relation to characteristics of individuals in which a biological condition is present and that may aid in the treatment of the individuals with respect to the biological condition. The integrated data repository may be generated and organized by analyzing and modifying raw health insurance claims data in a manner that enables the data stored by the integrated data repository to be further analyzed to determine trends, characteristics, features, and/or insights with respect to individuals in which one or more biological conditions may be present. For example, health insurance codes may be stored in the integrated data repository in such a way that at least one of medical procedures, biological conditions, treatments, dosages, manufacturers of medications, distributors of medications, or diagnoses may be determined for a given individual based on health insurance claims data for the individual. In various examples, the data integration and analysis system may generate and implement one or more tables that indicate correlations between health insurance claims data and various treatments, symptoms, or biological conditions that correspond to the health insurance claims data. Further, the integrated data repository may be generated using genomics data records of the group of individuals. In various examples, the large amounts of health insurance claims data may be matched with genomics data for the group of individuals to generate the integrated data repository.

By integrating the genomics data records for the group of individuals with the health insurance claims records, the data integration and analysis system may determine correlations between the presence of one or more biomarkers that are present in the genomics data records with other characteristics of individuals that are indicated by the health insurance claims data records that existing systems are typically unable to determine. For example, the data integration and analysis system may determine one or more genomic characteristics of individuals that correspond to treatments received by individuals, timing of treatments, dosages of treatments, diagnoses of individuals, smoking status, presence of one or more biological conditions, presence of one or more symptoms of a biological condition, one or more combinations thereof, and the like. Based on the correlations determined by the data integration and analysis system using the integrated data repository, cohorts of individuals that may benefit from one or more treatments may be identified that would not have been identified in existing systems. In one or more examples, the processes and techniques implemented to integrate the health insurance claims records and the genomics claims records in order to generate the integrated data repository may be complex and implement efficiency-enhancing techniques, systems, and processes in order to minimize the amount of computing resources used to generate the integrated data repository.

In one or more illustrative examples, the data pipeline system may access information stored by the integrated data repository to generate datasets that include a number of additional data records that include information related to at least a portion of the group of individuals. In an illustrative example, the additional data record includes information indicating whether individuals are included in a cohort of individuals in which lung cancer is present. The data pipeline system may execute a plurality of different sets of data processing instructions to determine a cohort of the group of individuals in which lung cancer is present. In various examples, the additional data record may indicate information used to determine a status of an individual with respect to lung cancer, such as one or more transaction insurance identifier, one or more international classification of diseases (ICD) codes, and one or more health insurance transaction dates. In addition to including a column that indicates whether an individual is included in the lung cancer cohort, the additional data record may include a column indicating a confidence level of the status of the individual with respect to the presence of lung cancer.

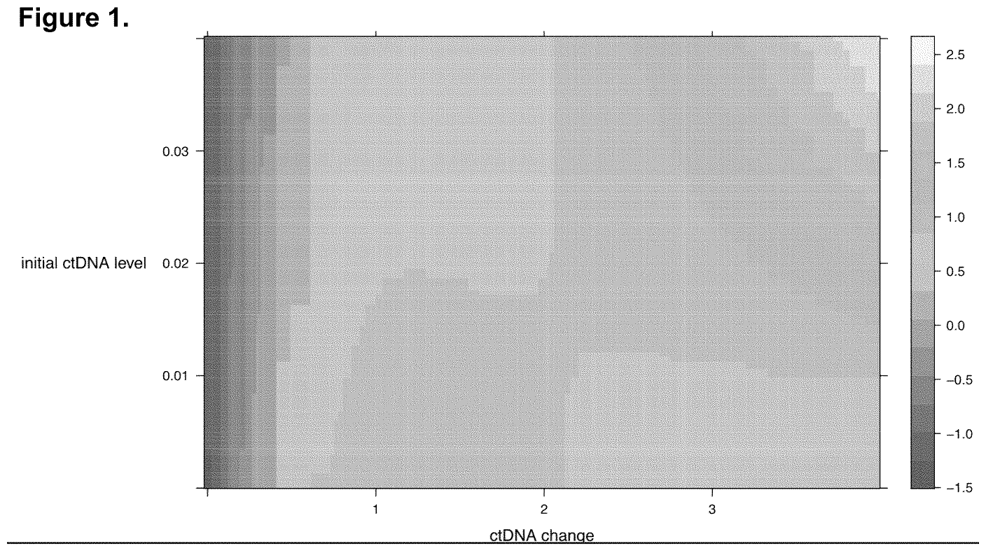

Described herein is a schematic diagram of a computing architecture 600 to incorporate medical records data into an integrated data repository. In various examples, at least a portion of the operations of the computing architecture may be performed by the data integration and analysis system of FIGS. 1, 3, and 4. In one or more examples, at least a portion of the operations of the computing architecture may be performed by one or more additional computing systems that are at least one of controlled, maintained, or implemented by a service provider that also at least one of controls, maintains, or implements the data integration and analysis system. In one or more additional examples, at least a portion of the operations of the computing architecture may be performed by a number of servers in a distributed computing environment.

The computing architecture may include a medical records data repository. The medical records data repository may store medical records data from a number of individuals. The medical records data may include imaging information, laboratory test results, diagnostic test information, clinical observations, dental health information, notes of healthcare practitioners, medical history forms, diagnostic request forms, medical procedure order forms, medical information charts, one or more combinations thereof, and so forth. In various examples, for a given individual, the medical records data repository may store information obtained from one or more healthcare practitioners that is related to the individual.

The computing architecture may perform operation that includes obtaining data packages from the medical records data repository. In one or more examples, the data packages may be obtained in response to one or more requests sent to the medical records data repository for medical records that correspond to one or more individuals. In one or more additional examples, the data packages may be obtained by the computing architecture using one or more application programming interface (API) calls. In one or more illustrative examples, a first data package, a second data package, up to an Nth data package may be obtained using the computing architecture. The individual data packages, may correspond medical records of a respective individual. For example, the first data package may include medical records of a first individual, the second data package may include medical records of a second individual, and the Nth data package may include medical records of a third individual.

Individual data packages, may include a number of components. In one or more examples, individual data packages, may include individual components that correspond to medical records from different healthcare providers. In one or more additional examples, the individual data packages, may include individual components that correspond to different parts of medical records that correspond to one or more healthcare providers. In an illustrative example the second data package may include a first component, a second component, up to an Nth component. In one or more illustrative examples, the first component may include a first portion of medical records of an individual, the second component may include a second portion of medical records of an individual, and the Nth component may include a third portion of medical records of an individual. In various examples, the first component may correspond to medical records of a first healthcare provider for the individual, the second component may correspond to medical records of a second healthcare provider for the individual, and the third component may correspond to medical records of a third healthcare provider for the individual. In one or more additional illustrative examples, the first component may include a first section of medical records of the individual, such as one or more forms related to a diagnostic test or procedure, and the second component may include a second section of medical records of the individual, such as a pathology report of the individual.

At operation, the computing architecture may preprocess individual data packages to identify a corpus of information to be analyzed. In one or more examples, the preprocessing of data packages obtained from the medical records data repository, may include transforming the data included in the data packages. For example, preprocessing the data packages may include transforming at least a portion of the data obtained from the medical records data repository to machine encoded information. To illustrate, preprocessing the data packages may include performing one or more optical character recognition (OCR) operations with respect to at least a portion of the data packages obtained from the medical records data repository. By converting at least a portion of the data packages obtained from the medical records data repository to machine encoded information, the data packages may be subjected to a number of operations, such as one or more parsing operations to identify one or more characters or strings of characters or one or more editing operations that are unable to be performed with respect to at least a portion of the data packages obtained from the medical records data repository.

In one or more examples, the preprocessing of individual data packages may include determining information included in individual data packages that is to be excluded from further analysis by the computing architecture. In various examples, one or more components of individual data packages may be excluded from a corpus of information to be analyzed. For example, with respect to the second data package, the computing architecture may determine that the first component is to be excluded from further analysis by the computing architecture. In one or more examples, the computing architecture may analyze the components, and/or with respect to one or more keywords to identify at least one of the components, and/or to exclude from further analysis by the computing architecture. In one or more illustrative examples, the computing architecture may parse the components, and/or to identify one or more keywords and in response to identifying the one or more keywords in a component, and/or, the computing architecture may determine to exclude the respective component, and/or from further analysis by the computing architecture. For example, the computing architecture may determine that the first component of the second data package is a test requisition form for one or more diagnostic procedures or tests. In these scenarios, the computing architecture may determine that the first component is to be excluded from further analysis by the computing architecture. Additionally, the computing architecture may determine that at least one of the second component and/or correspond to one or more pathology reports for an individual based on one or more keywords included in at least one of the second component or the Nth component. In these instances, the computing architecture may determine that at least a portion of the second component and/or at least a portion of the Nth component is to be included in the corpus of information to be further analyzed by the computing architecture.

In addition, a subset of the components of individual data packages obtained from the medical records data repository may be included in the corpus of information. In various examples, one or more additional operations may be performed to narrow the corpus of information. For example, one or more queries may be applied to a subset of information obtained from the medical records data repository. The one or more queries may extract information from the one or more data packages that satisfy the one or more queries. In at least some examples, the one or more queries may be a group of queries that are applied to individual components of a data package. In one or more illustrative examples, the group of queries may determine information to be included in the corpus of information and additional information that is to be excluded from the corpus of information. In one or more additional examples, one or more sections of at least one component of a data package may be excluded from the corpus of information.

In one or more additional illustrative examples, after determining that the first component is to be excluded from further analysis by the computing architecture, the computing architecture may then cause one or more queries to be implemented with respect to at least one the second component or the Nth component. In these scenarios, the one or more queries may determine that a section of the second component, such as a section that indicates family history for one or more biological conditions, is to be excluded from the corpus of information. In various examples, the one or more queries may be directed to identifying a number of keywords and/or combinations of keywords included in at least one of the second component or the Nth component. In these instances, the computing architecture may exclude from the corpus of information one or more portions of the individual components of the data packages that include one or more keywords or combinations of keywords. In one or more additional examples, the computing architecture may exclude from the corpus of information a number of words, a number of characters, and/or a number of symbols following one or more keywords that are included in one or more portions of the individual components of the data packages.

Further, at operation, the computing architecture may analyze the corpus of information to determine characteristics of individuals. In one or more examples, the computing architecture may analyze the corpus of information to determine individuals that have one or more phenotypes. In various examples, the computing architecture may analyze the corpus of information to determine one or more biomarkers that are indicative of a biological condition. For example, the computing architecture may analyze the corpus of information to determine individuals having one or more genetic characteristics. The one or more genetic characteristics may include at least one of one or more variants of a genomic region that correspond to a biological condition. In one or more illustrative examples, the one or more genetic characteristics may correspond to one or more variants of a genomic region that correspond to a type of cancer. In one or more additional illustrative examples, the one or more biomarkers may correspond to levels of an analyte being outside of a specified range. To illustrate, the computing architecture may analyze the corpus of information to determine individuals having levels of one or more proteins and/or levels of one or more small molecules present that are indicative of a biological condition. In these scenarios, the computing architecture may analyze results of laboratory tests to determine levels of analytes of individuals. In one or more additional examples, the computing architecture may analyze the corpus of information to determine individuals in which one or more symptoms are present that are indicative of a biological condition. In one or more further examples, the computing architecture may analyze imaging information included in the corpus of information to determine individuals in which one or more biomarkers are present.

In one or more examples, the computing architecture may implement one or more machine learning techniques to analyze the corpus of information. For example, the computing architecture may implement one or more artificial neural networks, such as at least one of one or more convolutional neural networks or one or more residual neural networks to analyze the corpus of information. The computing architecture may also implement at least one of one or more random forests techniques, one or more hidden Markov models, or one or more support vector machines to analyze the corpus of information.

In at least some implementations, the computing architecture may analyze the corpus of information by performing one or more queries with respect to the corpus of information. The one or more queries may correspond to one or more keywords and/or combinations of keywords. The one or more keywords and/or combinations of keywords may correspond to at least one of characters or symbols that correspond to one or more biological conditions. To illustrate, a keyword may correspond to characters related to a mutation of a genomic region, such as HER2. In one or more additional illustrative examples, one or more criteria may be associated with combinations of keyworks. To illustrate, a criterion that corresponds to a combination of keywords may include a number of words being present within a specified distance of one another in a portion of the corpus of information for an individual, such as the words fatigue, blood pressure, and swelling occurring within characters of one another. In these instances, the computing architecture may parse the corpus of information for the one or more keywords and/or combinations of keywords. In various examples, in response to determining that the one or more keywords and/or combinations of keywords are present in accordance with one or more criteria, the computing architecture may determine that a biological condition is present with respect to a given individual.

In one or more additional examples, the one or more queries may be image-based and the computing architecture may analyze images included in the corpus of information with respect to template images. The template images may be generated based on analyzing a number of images in which a biological condition is present and aggregating the number of images into a template image. In these scenarios, the computing architecture may analyze images included in the corpus of information with respect to one or more template images to determine a measure of similarity between the images included in the corpus of information and the template images. In situations where the measure of similarity for an individual is at least a threshold value, the computing architecture may determine that a characteristic of a biological condition is present in the individual.

After determining individuals having one or more characteristics, the computing architecture may, at operation, generate data structures that store data for individuals having the one or more characteristics. In one or more examples, the computing architecture may generate data tables that indicate individuals having an individual characteristics and/or individuals having a group of characteristics. For example, the computing architecture may generate a first data table and a second data table. The first data table may indicate individuals having one or more first characteristics and the second data table may indicate individuals having one or more second characteristics. In one or more illustrative examples, the first data table may indicate individuals having one or more first biomarkers for a biological condition and the second data table may indicate individual having one or more second biomarkers for the biological condition. The one or more first biomarkers may correspond to one or more first genomic variants that are associated with the biological condition and the one or more second biomarkers may correspond to one or more second genomic variants that are associated with the biological condition. In various examples, the data tables, may indicate whether or not the one or more characteristics associated with the individual data tables, are present with respect to individual individuals. To illustrate, the first data table may include a first indication for individuals in which one or more first genomic variants are present and a second indication for individuals in which the one or more first genomic variants are not present. In one or more additional examples, the first data table may indicate smoking status of individuals and the second data table may indicate whether or not individual individuals have received one or more treatments for a biological condition.

In one or more illustrative examples, the first data table and the second data table may have rows that correspond to individual individuals. In at least some examples, an individual identifier may be present in individual rows. The individual identifier may include at least one of alphanumeric characters or symbols that correspond to an individual. In various examples, the individual identifier may be present in a data package that corresponds to an individual. Columns of the first data table and the second data table may indicate a status of individual individuals with respect to one or more characteristics. For example, the columns of the data tables, may include an identifier that includes at least one of alphanumeric characters or symbols that indicate the presence or absence of one or more characteristics for a given individual. Further, although the illustrative example of includes a first data table and a second data table, the computing architecture may generate more data tables or fewer data tables.

At operation, the computing architecture may store the data structures in an additional data repository. For example, the computing architecture may store at least the first data tale and/or the second data table in an intermediate data repository. In various examples, the first data table and the second data table may be temporarily stored in the intermediate data repository. In one or more illustrative examples, the first data table and the second data table may be stored in the intermediate data repository before being added to the integrated data repository. In one or more examples, the integrated data repository may be periodically generated and/or updated. In these scenarios, data structures generated by the computing architecture based on analyzing the corpus of information may be stored in the intermediate data repository until a time when the integrated data repository is to be at least one of generated or updated.

Prior to adding data structures stored by the intermediate data repository to the integrated data repository, the computing architecture may perform one or more de-identification processes at operation. The data structures stored by the intermediate data repository may be de-identified in order to preserve the privacy of individuals. The one or more de-identification processes may include applying one or more electronically implemented cryptographic techniques to information of individuals included in the data structures stored by the intermediate data repository. In one or more examples, the computing architecture may generate tokens that correspond to individual individuals that have information stored in data structures of the intermediate data repository. The tokens may be generated by applying one or more hash functions to information related to individual individuals. In one or more examples, the one or more de-identification processes may include applying a salt function to information corresponding to individual individuals to generate tokens for the individual individuals. In various examples, the one or more cryptographic techniques applied to de-identify the data structures stored by the intermediate data repository may be the same or similar to those applied to information obtained from the health insurance claims data repository.

At operation, the computing architecture may store the de-identified data structures in conjunction with the integrated data repository. For example, the information stored in the intermediate data repository for a given individual may be stored in conjunction with additional information about the given individual in the integrated data repository. To illustrate, the integrated data repository may store information for a given individual obtained from at least two of the molecular data repository, obtained from the health insurance claims data repository, and obtained from the intermediate data repository. In this way, information about a given individual obtained from a number of disparate data repositories may be stored in the integrated data repository. As a result, information about individuals that is obtained from the different data repositories may be analyzed together rather than analyzed separately as with many existing systems.

In various examples, the information stored by the intermediate data repository may be used to validate one or more determinations made by the data integration and analysis system. For example, the data integration and analysis system may analyze information obtained from the health insurance claims data repository and the molecular data repository to determine characteristics of individuals. The data integration and analysis system may then analyze information obtained from the intermediate data repository to determine whether the predicted characteristics identified from the information obtained from the health insurance claims data repository and from the molecular data repository correspond to the characteristics for the same individuals with respect to information stored by the intermediate data repository.

The one or more cryptographic techniques applied to de-identify the data structures stored by the intermediate data repository may utilize the same or similar information that was used to generate at least one of the first tokens or the second tokens. For example, the operation may implement one or more cryptographic techniques using a combination of at least a portion of a first name of the respective individuals, at least a portion of the last name of the respective individuals, at least a portion of a date of birth of the respective individuals, a gender of the individuals, and at least a portion of a location identifier of the respective individuals to de-identify the data structures of the intermediate data repository. By utilizing the same or similar cryptographic techniques and the same or similar subset of information to de-identify the data structures stored by the intermediate data repository as were used to generate at least one of the first tokens or the second tokens, the information stored by the intermediate data repository may be synchronized with information for the same individuals that have information stored in the integrated data repository. Both the integrated data repository and the intermediate data repository may store information for thousands, tens of thousands, up to millions of individuals. Thus, without the ability to synchronize the individuals having records stored by the integrated data repository and the intermediate data repository through the use of a specified cryptographic protocol as described herein, the data structures of the integrated data repository and the data structures of the intermediate data repository that are associated with a same individual may not be stored in a manner such that the information stored by the integrated data repository and the information stored by the intermediate data repository may be retrieved together for a given individual, which may lead to inaccurate information being provided by the data integration and analysis system. The absence of a specified cryptographic protocol as described herein may also lead to the use of more computing resources to determine the information stored in the integrated data repository from other data sources and the information stored by the intermediate data repository that correspond to a given individual. FIGS. 7 and 8 illustrate example processes to generate an integrated data repository and generate datasets used in the analysis of information stored by the integrated data repository. The example processes are illustrated as collections of blocks in logical flow graphs, which represent sequences of operations that may be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks may be combined in any order and/or in parallel to implement the process.

Described herein is a data flow diagram of an example process to generate an integrated data repository that stores health insurance claims data and genomics data, according to one or more implementations. At operation, the process may include generating a data file that includes tokens generated using a first hash function. Individual tokens may correspond to a respective individual of a group of individuals having data stored by a molecular data repository. In one or more examples, an individual having data stored by the molecular data repository may be associated with one or more tokens. The tokens may be generated by applying one or more first hash functions to a subset of information corresponding to the group of individuals stored by the genomics data repository. In various examples, individual tokens may be generated by applying one or more first hash functions to one or more combinations of at least a portion of a first name of a respective individual of the group of individuals, at least a portion of a second name of a respective individual of the group of individuals, a location identifier of a respective individual of the group of individuals, a gender of a respective individual of the group of individuals, and a date of birth of a respective individual of the group of individuals. In one or more illustrative examples, the tokens may be generated by a data integration and analysis system that is coupled to the genomics data repository. In one or more additional illustrative examples, the tokens may be generated by a third-party system and accessed by a data integration and analysis system coupled to the molecular data repository. The process may also include, at operation, sending the data file to a health insurance claims data management system. The health insurance claims data management system may match the tokens included in the data file with second tokens accessed by the health insurance data management system and generated based on information stored by a health insurance claims data repository.

In addition, at operation, the process may include obtaining, from the health insurance claims data management system, in response to the data file, first data corresponding to the group of individuals, where the first data includes health insurance claims data. In some implementations, affirmative consent is obtained from the members of the group of individuals for their data to be transferred from the health insurance claims data management system. In one or more examples, the data is transferred in an anonymized format, such that the data may not be traced back to an individual member. The health insurance claims data management system may be coupled to a health insurance claims data repository that stores health insurance claims information for a number of individuals. In one or more examples, the health insurance claims data management system may analyze the tokens of the data file with respect to additional tokens generated by the health insurance claims data management system. The additional tokens may be generated based on a same set of information used to generate the tokens included in the data file. However, an individuals identity may not be determined based on a token. In various examples, the health insurance claims data management system may match tokens included in the data file with additional tokens generated based on information stored by the health insurance claims data repository to determine individuals having information stored by the health insurance claims data repository that also have information stored by the genomics data repository. The technology disclosed herein complies with legal and best practice privacy standards, such as HIPAA and GDPR.

At operation, the process may include generating a number of identifiers using a second hash function that is different from the first hash function. In one or more examples, individual identifiers may correspond to one or more tokens related to a respective individual of the group of individuals. The identifiers may be unique with respect to a given individual of the group of individuals and are de-identified. Additionally, the identifiers may be generated using information stored by the genomics data repository for the group of individuals that is different from the information stored by the genomics data repository used to generate the tokens. In various examples, intermediate identifiers may be generated by applying the second hash function to information of the respective groups of individuals and final versions of the identifiers may be generated by applying one or more salting techniques to the intermediate identifiers. Information stored by the genomics data repository for respective individuals may be stored in association with the identifiers such that at least a portion of the information for given individuals stored by the genomics data repository may be accessed using respective identifiers of the given individuals.

Further, the process may include, at operation, obtaining, using the number of identifiers, second data from the molecular data repository for the group of individuals, and, at operation, the process may include determining respective portions of the first data that correspond to respective portions of the second data for the group of individuals. For example, for a given individual, the first data corresponding to health insurance claims data for the given individual may be identified in addition to second data corresponding to molecular data of the given individual, such as genomics data. In this way, for a given individual, both health insurance claims data and molecular data may be identified.

The process may include, at operation, generating an integrated data repository that stores the respective portions of the first data and the respective portions of the second data in relation to respective identifiers of the number of identifiers. For example, the integrated data repository may store health insurance claims data and genomics claims data for a given individual in association with an identifier that may be used to access the health insurance claims data and the genomics claims data for the given individual. The information stored by the integrated data repository may be organized according to a data repository schema. For example, the integrated data repository may store health insurance claims data and genomics data for the group of individuals in a number of data tables. In one or more examples, information stored by the number of data tables may be linked. To illustrate, information related to a given individual stored by a first data table of the data repository schema may be linked to additional information related to the given individual stored by a second data table of the data repository schema. In this way, information accessed in one data table of the data repository schema may result in accessing additional information stored in another data table of the data repository schema.

In one or more illustrative examples, the data repository schema may include a first data table that stores genomics data of the group of individuals. For example, the first data table may store information corresponding to a panel used to generate genomics data, mutations of genomic regions, types of mutations, copy numbers of genomic regions, coverage data indicating numbers of nucleic acid molecules identified in a sample having one or more mutations, testing dates, and patient information. The data repository schema may also include a second data that stores data related to one or more patient visits by individuals to one or more healthcare providers and a third data table that stores information corresponding to respective services provided to individuals with respect to one or more patient visits to one or more healthcare providers indicated by the second data table. Additionally, the data repository schema may include a fourth data table that stores personal information of the group of individuals and a fifth data table that stores information related to a health insurance company or governmental entity that made payment for services provided to the group of individuals. Further, the data repository schema may include a sixth data table storing information corresponding to health insurance coverage information for the group of individuals, such as a type of health insurance plan related to the group of individuals. The data repository schema may also include a seventh data table that stores information related to pharmaceutical treatments obtained by the group of individuals.

In one or more examples, the integrated data repository may also store medical records that correspond to at least a portion of the group of individuals. In these examples, the medical records may be obtained from one or more data repositories storing the medical records. One or more optical character recognition (OCR) operations may be performed with respect to the medical records. Additionally, the medical records may be analyzed to determine one or more portions of the additional information to remove to produce a corpus of information. In various examples, the corpus of information may be analyzed to determine a portion of the subset of the additional group of individuals that correspond to one or more biomarkers.

One or more data structures may be generated from the corpus of information that store identifiers of the portion of the subset of the additional group of individuals and that store an indication that the portion of the subset of the additional group of individuals corresponds to the one or more biomarkers. The one or more data structures may be stored by an intermediate data repository. One or more de-identification operations may be performed with respect to the identifiers of the portion of the subset of the additional group of individuals before modifying the integrated data repository to store at least a portion of the additional information of the medical records of the portion of the subset of the additional group of individuals in relation to the number of identifiers. After de-identification of the information stored by the one or more data structures, the information stored by the integrated data repository may be added to the integrated data repository. In at least some examples, the de-identified medical records information may be added to the integrated data repository in addition to or in lieu of the health insurance claims data. In various examples, the one or more data structures storing the de-identified medical records information with respect to the biomarker data may have one or more logical connections with other data structures stored in the integrated data repository. To illustrate, the one or more data structures storing the de-identified medical records information with respect to the biomarker data may have one or more logical connections with at least one of the first data table may store information corresponding to a panel used to generate genomics data, mutations of genomic regions, types of mutations, copy numbers of genomic regions, coverage data indicating numbers of nucleic acid molecules identified in a sample having one or more mutations, testing dates, and patient information, the second data that stores data related to one or more patient visits by individuals to one or more healthcare providers, the a third data table that stores information corresponding to respective services provided to individuals with respect to one or more patient visits to one or more healthcare providers indicated by the second data table, the fourth data table that stores personal information of the group of individuals, the fifth data table that stores information related to a health insurance company or governmental entity that made payment for services provided to the group of individuals, the sixth data table storing information corresponding to health insurance coverage information for the group of individuals, such as a type of health insurance plan related to the group of individuals, or the seventh data table that stores information related to pharmaceutical treatments obtained by the group of individuals.

In various examples, the medical records data may be added to the integrated data repository by generating a data file including the first tokens generated using a first hash function. Individual first tokens may correspond to a respective individual of a group of individuals having data stored by a molecular data repository. Additionally, the data file may be sent to a medical records data management system and medical records data corresponding to the group of individuals may be obtained from the medical records data management system in response to the data file. Further, a number of identifiers may be generated using a second hash function that is different from the first hash function. Each identifier may correspond to one or more tokens related to each individual of the group of individuals. Using the number of identifiers second data may be obtained from the molecular data repository for the group of individuals. In various examples, respective portions of the first data may be determined to correspond to respective portions of the second data for the group of individuals. In this way, the integrated data repository may be generated that stores the respective portions of the first data and the respective portions of the second data in relation to respective identifiers of the number of identifiers.

After the integrated data repository storing medical records data is generated, a request may be received to determine data with respect to a number of individuals having data stored in the integrated data repository. The request includes one or more search criteria. In one or more examples, a subset of the number of individuals having one or more characteristics that correspond to the one or more search criteria may be determined and information of the subset of the number of individuals may be analyzed to determine a measure of significance of a characteristic of the one or more characteristics with respect to a biological condition.

In one or more illustrative examples, one or more genomic mutations may be determined to be present in the subset of the number of individuals and a plurality of treatments provided to the subset of the number of individuals may also be determined. In various examples, respective survival rates for the subset of the number of individuals may be determined, such as real-world survival rates. In at least some examples, the measure of significance may correspond to survival rate with respect to a treatment of the plurality of treatments and a genomic mutation of the one or more genomic mutations. Based on measure of significance, an effectiveness of the treatment for the subset of the number of individuals may be determined. In one or more examples, individuals in subset of the number of individuals that have not received the treatment may be determined. One or more therapeutically effective amounts of the treatment may be administered to the individuals in the subset of the number of individuals that have not received the treatment.

Described herein is a data flow diagram of an example process to generate a number of datasets used to analyze information stored by an integrated data repository that stores health insurance claims data and genomics data, according to one or more implementations. The process may include, at operation, determining a first set of data processing instructions that are executable in relation to the first data stored by an integrated data repository. The integrated data repository may store health insurance claims data and molecular data for a common group of individuals. In one or more examples, the first set of data processing instructions may be included in a plurality of sets of data processing instructions that are part of a data processing pipeline. Each of the sets of data processing instructions of the data processing pipeline may be executed to generate a respective analytics ready dataset. For example, individual sets of data processing instructions of the data processing pipeline may be executable to generate datasets that include specified portions of information and/or combinations of information stored by the integrated data repository. In one or more additional examples, individual sets of data processing instructions of the data processing pipeline may be executable to analyze and modify portions of information stored by the integrated data repository to generate respective datasets. Additionally, individual sets of data processing instructions may be executable with respect to individual subsets of information stored by the integrated data repository.

The process may also include, at operation, causing the first set of data processing instructions to be executed to generate a first dataset. The first dataset may indicate a subset of the group of individuals in which a biological condition is present. The first set of data processing instructions may be executed to analyze data stored by the integrated data repository to identify a cohort of individuals in which the biological condition is present. In one or more illustrative examples, the biological condition may include a cancer. To illustrate, the first set of data processing instructions may be executed to analyze data stored by the integrated data repository to identify a cohort of individuals in which lung cancer is present. In various examples, the data processing pipeline may include multiple sets of data processing instructions to identify cohorts of individuals in which different biological conditions are present.

In one or more examples, the first set of data processing instructions may be executed to analyze at least one of health insurance claims data or molecular data to determine a cohort of individuals in which the biological condition is present. For example, the first set of data processing instructions may be executed to identify individuals having one or more health insurance codes present in health insurance claims data to determine a group of individuals in which the biological condition is present. Additionally, the first set of data processing instructions may be executed to identify individuals in which one or more mutations are present in a genomic region of nucleic acid molecules derived from samples obtained from the individuals to determine a group of individuals in which the biological condition is present.

In addition, the process may include, at operation, determining a second set of data processing instructions that are executable in relation to second data stored by the integrated data repository. The second set of data stored by the integrated data repository may be different from the first set of data stored by the integrated data repository and analyzed in relation to the first set of data processing instructions. For example, the first data may correspond to first columns of one or more first data tables stored by the integrated data repository and the second data may correspond to second columns of one or more second data tables stored by the integrated data repository.

At operation, the process may include causing the second set of data processing instructions to be executed to generate a second dataset indicating one or more treatments provided to a second subset of the group of individuals. The second dataset may indicate a subset of the group of individuals that have received one or more treatments. The one or more treatments may be provided to individuals in which one or more biological conditions are present. In one or more examples, the second set of data processing instructions may be executed to analyze data stored by the integrated data repository to identify a cohort of individuals that received the one or more treatments. To illustrate, the second set of data processing instructions may be executed to analyze at least one health insurance claims data or genomics data to determine a cohort of individuals that received the one or more treatments. In one or more illustrative examples, the second set of data processing instructions may be executed to identify individuals having one or more health insurance codes present in health insurance claims data to determine a group of individuals that received the one or more treatments.

Further, the process may include, at operation, determining a third subset of the group of individuals that includes a portion of the first subset of the group of individuals that overlaps with a portion of the second subset of the group of individuals. As a result, the third subset of the group of individuals corresponds to individuals in which both the biological condition is present and the one or more treatments are provided. At, the process, may include analyzing the first dataset and the second dataset with respect to the third subset of the group of individuals to determine a measure of significance of a characteristic of the third subset of the group of individuals. In one or more examples, one or more machine learning techniques or statistical techniques may be applied to information included in at least one of the first dataset and the second dataset with respect to the third subset of the group of individuals. The measure of significance may correspond to a statistical measure of significance with respect to the characteristic. In one or more additional examples, the measure of significance may correspond to a probability of the characteristic being present in individuals in which the biological condition is present.

In one or more illustrative examples, the characteristic may include one or more treatments provided to the individuals in which the biological condition is present. In one or more additional illustrative examples, the characteristic may include the presence of a mutation of a genomic region of nucleic acid molecules derived from samples obtained from individuals in which the biological condition is present. In various examples, information included in at least one of the first dataset or the second dataset may be analyzed to determine an impact of the characteristic with respect to one or more metrics. In one or more examples, information included in at least one of the first dataset or the second dataset may be analyzed to determine an amount of influence of a treatment on a survival rate of individuals in which the biological condition is present. In one or more further examples, information included in at least one of the first dataset or the second dataset may be analyzed to determine an amount of influence of a mutation of a genomic region on a survival rate of individuals in which the biological condition is present. Additionally, information included in the first dataset and the second dataset may be analyzed to determine an amount of impact of one or more treatments with respect to individuals in which the biological condition is present and in which one or more genomic mutations are also present.

Described herein is a machine in the form of a computer system within which a set of instructions may be executed for causing the machine to perform any one or more of the methodologies discussed herein, according to an example, according to an example implementation. For example, a machine in the example form of a computer system, within which instructions (e.g., software, a program, an application, an applet, an app, or other executable code) for causing the machine to perform any one or more of the methodologies discussed herein may be executed. For example, the instructions may cause the machine to implement the architectures and frameworks described previously, and to execute the methods described with respect to previously.

The instructions transform the general, non-programmed machine into a particular machine programmed to carry out the described and illustrated functions in the manner described. In alternative implementations, the machine operates as a standalone device or may be coupled (e.g., networked) to other machines. In a networked deployment, the machine may operate in the capacity of a server machine or a client machine in a server-client network environment, or as a peer machine in a peer-to-peer (or distributed) network environment. The machine may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions, sequentially or otherwise, that specify actions to be taken by the machine. Further, while only a single machine is illustrated, the term “machine” shall also be taken to include a collection of machines that individually or jointly execute the instructions to perform any one or more of the methodologies discussed herein.

Examples of computing devices may include logic, one or more components, circuits (e.g., modules), or mechanisms. Circuits are tangible entities configured to perform certain operations. In an example, circuits may be arranged (e.g., internally or with respect to external entities such as other circuits) in a specified manner. In an example, one or more computer systems (e.g., a standalone, client or server computer system) or one or more hardware processors (processors) may be configured by software (e.g., instructions, an application portion, or an application) as a circuit that operates to perform certain operations as described herein. In an example, the software may reside (1) on a non-transitory machine readable medium or (2) in a transmission signal. In an example, the software, when executed by the underlying hardware of the circuit, causes the circuit to perform the certain operations.

In an example, a circuit may be implemented mechanically or electronically. For example, a circuit may comprise dedicated circuitry or logic that is specifically configured to perform one or more techniques such as discussed above, such as including a special-purpose processor, a field programmable gate array (FPGA) or an application-specific integrated circuit (ASIC). In an example, a circuit may comprise programmable logic (e.g., circuitry, as encompassed within a general-purpose processor or other programmable processor) that may be temporarily configured (e.g., by software) to perform the certain operations. It will be appreciated that the decision to implement a circuit mechanically (e.g., in dedicated and permanently configured circuitry), or in temporarily configured circuitry (e.g., configured by software) may be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise, a general-purpose processor configured via software, the general-purpose processor may be configured as respective different circuits at different times. Software may accordingly configure a processor, for example, to constitute a particular circuit at one instance of time and to constitute a different circuit at a different instance of time.

In an example, circuits may provide information to, and receive information from, other circuits. In this example, the circuits may be regarded as being communicatively coupled to one or more other circuits. Where multiples of such circuits exist contemporaneously, communications may be achieved through signal transmission (e.g., over appropriate circuits and buses) that connect the circuits. In implementations in which multiple circuits arc configured or instantiated at different times, communications between such circuits may be achieved, for example, through the storage and retrieval of information in memory structures to which the multiple circuits have access. For example, one circuit may perform an operation and store the output of that operation in a memory device to which it is communicatively coupled. A further circuit may then, at a later time, access the memory device to retrieve and process the stored output. In an example, circuits may be configured to initiate or receive communications with input or output devices and may operate on a resource (e.g., a collection of information).

The various operations of method examples described herein may be performed, at least partially, by one or more processors that are temporarily configured (e.g., by software) or permanently configured to perform the relevant operations. Whether temporarily or permanently configured, such processors may constitute processor-implemented circuits that operate to perform one or more operations or functions. In an example, the circuits referred to herein may comprise processor-implemented circuits.

Similarly, the methods described herein may be at least partially processor implemented. For example, at least some or all of the operations of a method may be performed by one or processors or processor-implemented circuits. The performance of certain of the operations may be distributed among the one or more processors, not only residing within a single machine, but deployed across a number of machines. In an example, the processor or processors may be located in a single location (e.g., within a home environment, an office environment or as a server farm), while in other examples the processors may be distributed across a number of locations.

The one or more processors may also operate to support performance of the relevant operations in a “cloud computing” environment or as a “software as a service.”

(SaaS). For example, at least some of the operations may be performed by a group of computers (as examples of machines including processors), with these operations being accessible via a network (e.g., the Internet) and via one or more appropriate interfaces (e.g., Application Program Interfaces (APIs).)

Example implementations (e.g., apparatus, systems, or methods) may be implemented in digital electronic circuitry, in computer hardware, in firmware, in software, or in any combination thereof. Example implementations may be implemented using a computer program product (e.g., a computer program, tangibly embodied in an information carrier or in a machine readable medium, for execution by, or to control the operation of, data processing apparatus such as a programmable processor, a computer, or multiple computers).

A computer program may be written in any form of programming language, including compiled or interpreted languages, and it may be deployed in any form, including as a stand-alone program or as a software module, subroutine, or other unit suitable for use in a computing environment. A computer program may be deployed to be executed on one computer or on multiple computers at one site or distributed across multiple sites and interconnected by a communication network.

In an example, operations may be performed by one or more programmable processors executing a computer program to perform functions by operating on input data and generating output. Examples of method operations may also be performed by, and example apparatus may be implemented as, special purpose logic circuitry (e.g., a field programmable gate array (FPGA) or an application-specific integrated circuit (ASIC)).

The computing system may include clients and servers. A client and server are generally remote from each other and generally interact through a communication network. The relationship of client and server arises by virtue of computer programs running on the respective computers and having a client-server relationship with each other. In implementations deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., computing device) and software architectures that may be deployed in example implementations.

In an example, the computing device may operate as a standalone device or the computing device may be connected (e.g., networked) to other machines.

In a networked deployment, the computing device may operate in the capacity of either a server or a client machine in server-client network environments. In an example, computing device may act as a peer machine in peer-to-peer (or other distributed) network environments. The computing device may be a personal computer (PC), a tablet PC, a set-top box (STB), a mobile telephone, a web appliance, a network router, switch or bridge, or any machine capable of executing instructions (sequential or otherwise) specifying actions to be taken (e.g., performed) by the computing device. Further, while only a single computing device is illustrated, the term “computing device” shall also be taken to include any collection of machines that individually or jointly execute a set (or multiple sets) of instructions to perform any one or more of the methodologies discussed herein.

Example computing device may include a processor (e.g., a central processing unit CPU), a graphics processing unit (GPU) or both), a main memory and a static memory, some or all of which may communicate with each other via a bus. The computing device may further include a display unit, an alphanumeric input device (e.g., a keyboard), and a user interface (UI) navigation device (e.g., a mouse). In an example, the display unit, input device and UI navigation device may be a touch screen display. The computing device may additionally include a storage device (e.g., drive unit), a signal generation device (e.g., a speaker), a network interface device, and one or more sensors, such as a global positioning system (GPS) sensor, compass, accelerometer, or another sensor.

The storage device may include a machine readable medium on which is stored one or more sets of data structures or instructions (e.g., software) embodying or utilized by any one or more of the methodologies or functions described herein. The instructions may also reside, completely or at least partially, within the main memory, within static memory, or within the processor during execution thereof by the computing device. In an example, one or any combination of the processor, the main memory, the static memory, or the storage device may constitute machine readable media.

While the machine readable medium is illustrated as a single medium, the term “machine readable medium” may include a single medium or multiple media (e.g., a centralized or distributed database, and/or associated caches and servers) that configured to store the one or more instructions. The term “machine readable medium” may also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” may accordingly be taken to include, but not be limited to, solid-state memories, and optical and magnetic media. Specific examples of machine-readable media may include non-volatile memory, including, by way of example, semiconductor memory devices (e.g., Electrically Programmable Read-Only Memory

(EPROM), Electrically Erasable Programmable Read-Only Memory (EEPROM) and flash memory devices; magnetic disks such as internal hard disks and removable disks; magneto-optical disks; and CD-ROM and DVD-ROM disks.

The instructions may further be transmitted or received over a communications network 828 using a transmission medium via the network interface device 822 utilizing any one of a number of transfer protocols (e.g., frame relay, IP, TCP, UDP, HTTP, etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

As used herein, a component, may refer to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

### EXAMPLES

### Example 1—Methods

In accordance with techniques described herein, two rounds of dataset generation were used. Both datasets included patients with molecular algorithm results, which consists of comparing the mean VAF of an initial Guardant 360 test to the mean VAF of a genomic test (e.g., Guardant G360) test at a second timepoint (cit). The first dataset queried a real world evidence database, which comprises aggregated commercial payer health claims and de-identified records from over 225,000 individuals with comprehensive ctDNA testing. In particular, was analysis patients with NSCLC treated with immune checkpoint inhibitors (ICI) (monotherapy or in combination) who received a ctDNA test within 15 weeks prior to treatment initiation and a second test 3-15 weeks after treatment initiation were retrospectively evaluated (dataset 1).

### Example 2—Additional Methods

Subsequently, a second dataset was generated including additional newer records from a real world evidence database. In particular was analysis of patients with NSCLC treated with immune checkpoint inhibitors (ICI) (monotherapy or in combination) who received a ctDNA test within 15 weeks prior to treatment initiation and a second test 3-15 weeks after treatment initiation were retrospectively evaluated (dataset 2; ICI treatment cohort). In addition, patients with NSCLC treated with immune checkpoint inhibitors (Tyrosine Kinase inhibitors including gefitinib, erlotinib, afatinib, and osimertinib (monotherapy or in combination) who received a ctDNA test within 15 weeks prior to treatment initiation and a second test 3-15 weeks after treatment initiation were retrospectively evaluated (dataset 2; TKI treatment cohort).

Cox proportional hazards (CPH) were used for RW time to next treatment (TTNT) and time to treatment discontinuation (TTD) analyses. Gender, age, line of therapy (LOT) and comorbidities were included as covariates.

### Example 3—Modeling and Analysis

For the first dataset, ctDNA change from baseline to on-treatment was modeled as a continuous variable using a regularized cubic spline or analogous alternatives shown in Table 1. Baseline ctDNA level, using either maximum variant allele fraction (maxVAF) or mean variant allele fraction (meanVAF) at baseline sample was modeled as an interaction effect with the ctDNA change from above. Model validation and calibration were carried out via bootstrap resampling, ANOVA was used for multiple design hypothesis testing.

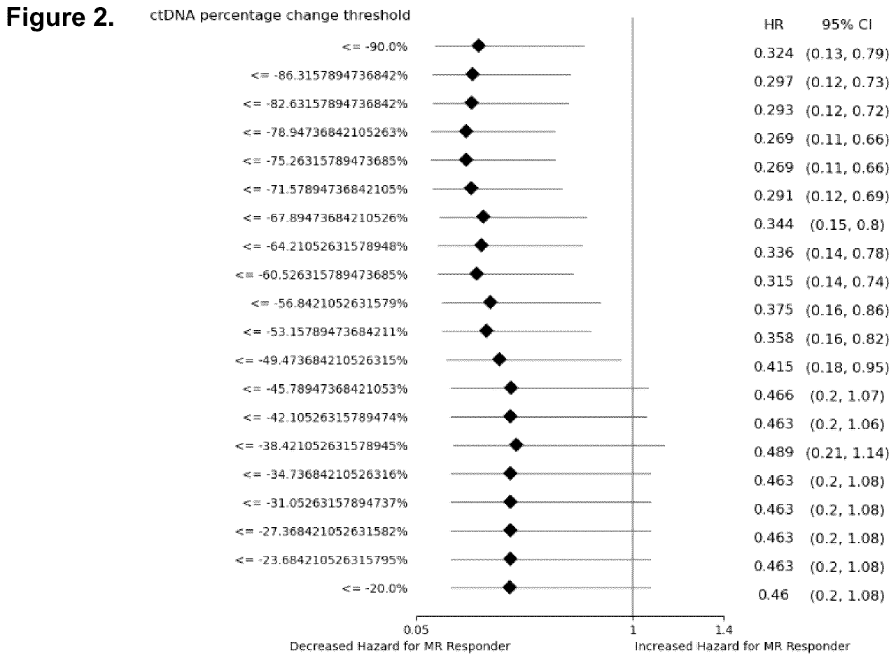

For the second dataset, CPH were also used for TTNT and overall survival (OS) analyses and ctDNA change from baseline to on-treatment was modeled as a categorical variable using a threshold of greater than or equal to 60% or 90% decrease in ctDNA for the ICI and TKI treatment cohorts respectively. Cutoffs were based on a sub analysis for optimal cut points for OS and TTNT in terms of hazard ratio and p-value from CPH analyses (see FIG. 2). Median TTNT and OS were calculated by Kaplan Meier.

### Example 4—Exemplary Illustrative Results

An initial cohort of 82 ICI treated patients from dataset 1 met the study criteria and had a Guardant molecular response algorithm ctDNA level change result of either “Decreasing” or “Increasing”. 31 patients with large ctDNA decrease (e.g. >=50% decrease), also known as molecular responders, had significantly longer TTNT and TTD compared to other patients, regardless of baseline ctDNA level. 51 patients with small ctDNA decrease or ctDNA increase showed ctDNA level dependent trends in TTNT and TTD, with decreased risk for shorter TTNT and TTD for lower baseline ctDNA level. We found that the interaction between baseline ctDNA level and ctDNA change was significant (TTNT: p=0.0069, TTD: p=0.0236), and that ctDNA change had a nonlinear effect on both TTNT and TTD (TTNT: p=0.0014, TTD: p=0.0349). TTNT (TTD) bootstrap-adjusted concordance indices were 0.69 (0.65) and hazard ratios [95% CI] were 5.36 [1.79, 16.10] (2.37 [1.13, 4.98]).

A follow up study was conducted on dataset 2 ICI treated and TKI treated cohorts. Similar as found in dataset 1, models of patient response that incorporated both baseline ctDNA level and ctDNA change.

### Example 5—Visualization of the CPH Model

As shown in FIG. 1, a representative visualization of the CPH model (utilizing splines for MR) for predicting whether a patient will have a TTNT event. The model is specified to use interaction between ctDNA change (using Guardant molecular response score; MR Score=mean treatment VAF/mean baseline VAF) and ctDNA level variable (mean baseline VAF). Plot shows how ctDNA change interacts with baseline and ctDNA level to affect the response in the CPH regression model.

Plot shows the relationship between the outcome (TTNT) and the explanatory variables (ctDNA change interacts with baseline and ctDNA level) while the other variables are held constant (weighted comorbidity score=21, female gender, patient age=66).

A notable relationships includes patients that are have high ctDNA level and high score for ctDNA change (large increase in ctDNA) have the shortest TTNT (progress on therapy and have a TTNT event). Also, patients with low ctDNA level and high score for ctDNA change have relatively long TTNT, suggesting that large relative changes in ctDNA do not lead to shorter TTNT in patients with low baseline ctDNA level. Patients with a low score for ctDNA change (large decrease in ctDNA) have the longest TTNT largely regardless of baseline ctDNA level. Thus the impact of baseline ctDNA level within the interaction model is most useful for patients with increasing ctDNA levels over timepoints.

### Example 6—Forest Plot of Hazard (HR) for Death from CPH for Molecular Responders

As shown in FIG. 2, a representative Forest plot of hazard (HR) for death from CPH for molecular responders, defined at varying thresholds or ctDNA change (from 90 decrease at top to 20% decrease at the bottom). This figure is for dataset 2 ICI cohort and −60% (60% decrease in ctDNA) was chosen as the optimal genomic MR threshold to define MR responder/non-responder for the ICI treated patient cohort. All the respective thresholds below were significant for TTNT. Figure shows significantly reduced HR for OS when patients achieved 50% or more reduction in ctDNA. In contrast to OS, all above thresholds were significant for longer rwTTNT (reduction of 20% or more in ctDNA).

### Example 7—Responder and Non-Responder

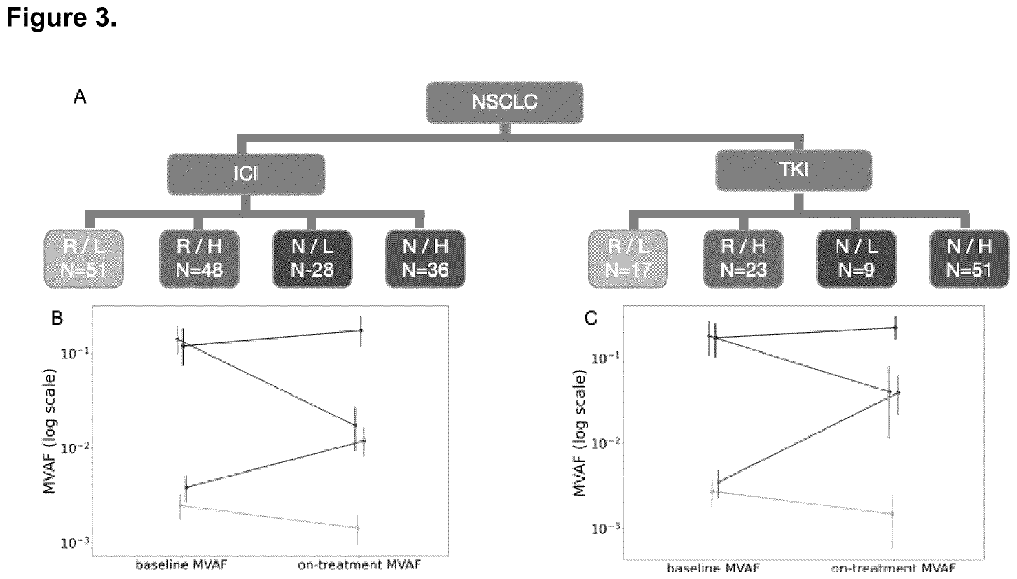

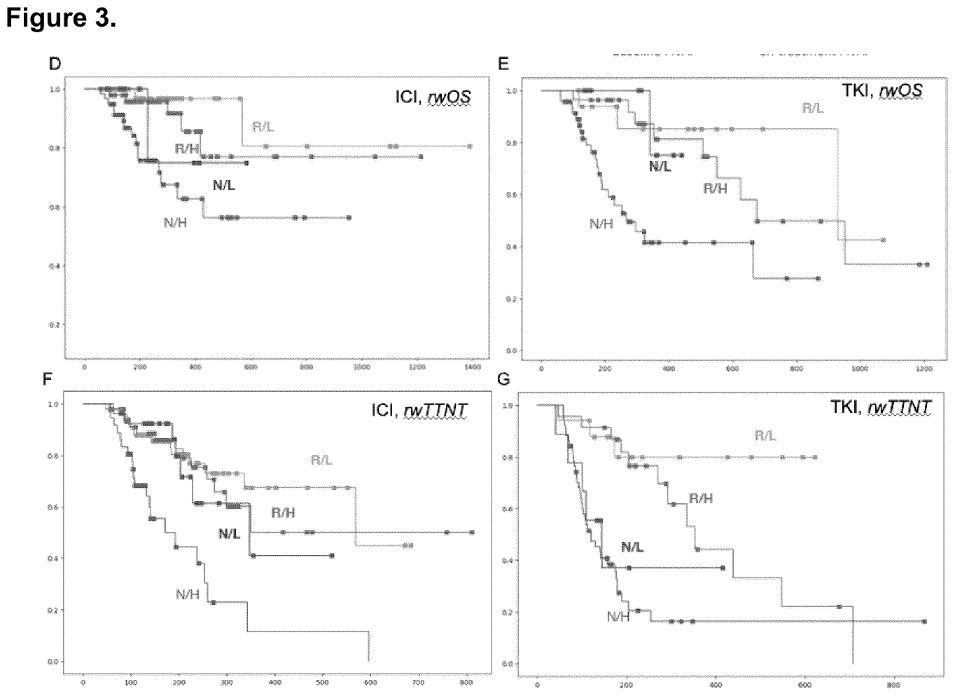

As shown in FIG. 3. Patients with “responder” and “non-responder” categories based on ctDNA change metric alone can be further sub divided by their baseline ctDNA level (high/low), resulting in 4 categories that better associate with both real world OS and TTNT. Non-responders with high levels of baseline ctDNA consistently have the shortest OS and TTNT in both the ICI and TKI treatment cohorts. A) Patients within ICI and TKI treatment cohorts were categorized into 4 categories: responder/low baseline (R/L), responder/high baseline (R/H), non-responder/low baseline (N/L), non-responder/high baseline (N/H) based on molecular responder (R)/non-responder (N) and high (H) or low (L) baseline ctDNA level. Patient counts and plots reflect thresholds optimized for rwTTNT individually. B-C) Maximum variant allele fraction (MVAF) is plotted (mean and 95% CI) between baseline and on-treatment timepoint for respective MR/MVAF groups within ICI cohort (B) and TKI cohort (C). Kaplan Meier plots of rwOS (D) and rwTTNT (F) in the ICI cohort and rwOS (E) and rwTTNT (G) in the TKI cohort. Example 1—One year survival probability for rwOS and rwTTNT

### Example 8—rwOS and rwTTNT

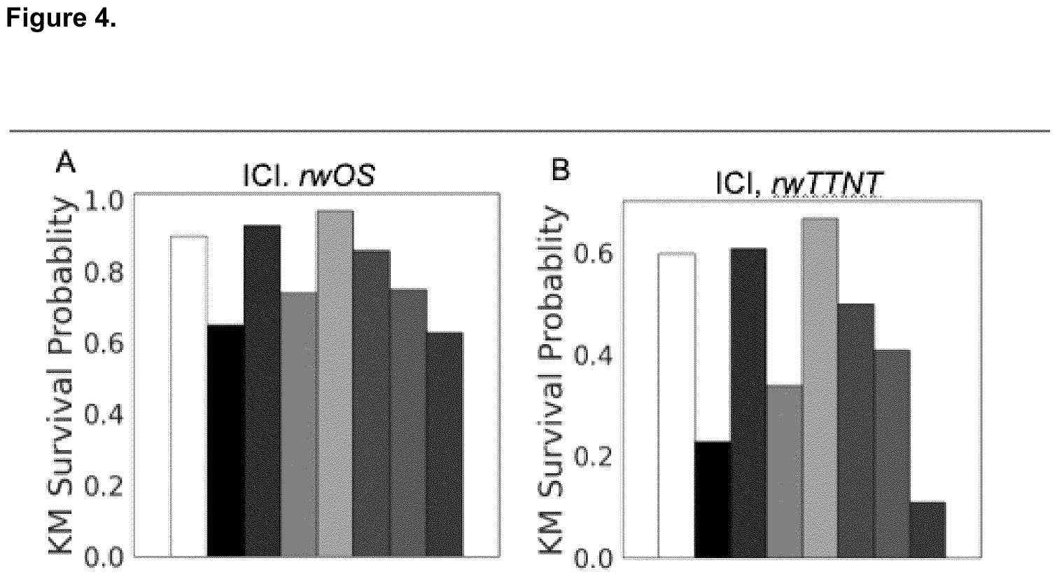

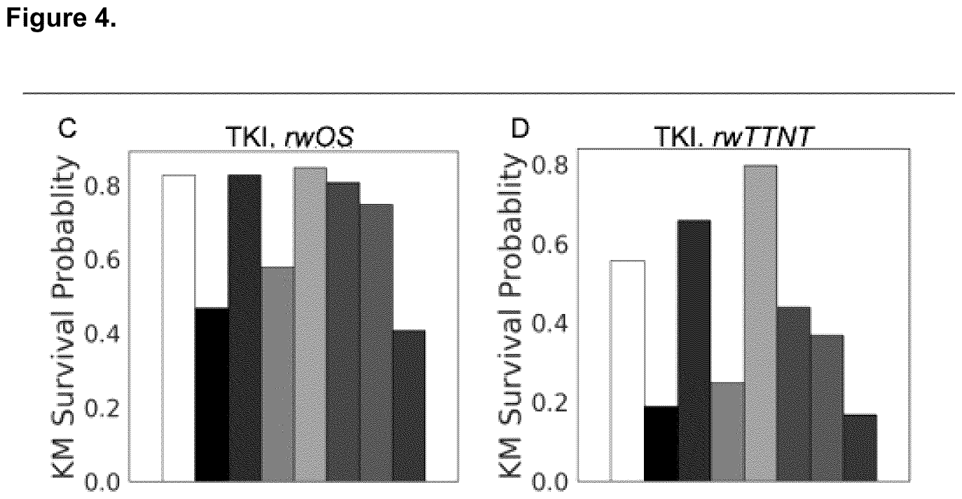

As shown in FIG. 4. One year survival probability for rwOS and rwTTNT for groups in FIG. 3. Patients with ctDNA change metric alone can be further sub divided by their baseline ctDNA level (high/low). Patients with low baseline ctDNA and decreasing in ctDNA change metric have higher percentage of patients without OS and TTNT event within 1 year, whereas patients with high baseline ctDNA and increasing in ctDNA change metric have lowest percentage of patients without OS and TTNT event within 1 year. Molecular responders (R, white) and nonresponders (N, black), low baseline ctDNA (L, maroon), high baseline ctDNA (H, grey), responder/low baseline (R/L, orange), responder/high baseline (R/H, green), non-responder/low baseline (N/L, red), non-responder/high baseline (N/H, blue) in ICI (A,B) and TKI cohorts (C,D).

### Example 8—Preliminary Findings

These data demonstrate a statistically significant interaction between baseline ctDNA level and treatment-induced ctDNA level change in association with clinical outcomes. Prediction of patient response to therapy has important use in clinical practice as well as investigational clinical trials. By incorporating metrics of baseline ctDNA level and ctDNA level change between timepoints and the interaction between these metrics, we demonstrate an improved model for predicting patient response to therapy. Although demonstrated for ICI-based and TKI-based therapies, we anticipate that predictive models of patient response such as ours will be applicable to cancer patients undergoing most types of therapy since decrease in ctDNA level is a biological measure of tumor burden and not specific to particular cancer treatments.

Table 3 shows that models that include baseline ctDNA level interaction with ctDNA change (MR in table) have superior performance in prediction/association with TTNT per concordance index and AIC over models with only ctDNA change. Covariates (cov) included in the analysis include LOT, gender, age, and a weighted comorbidity index. MR (>50% decrease) denotes the categorical variable after thresholding Guardant Response MR score by greater than or equal and less than 50% decrease in ctDNA between timepoints.

TABLE 3
TTNT outcome (36 events among 81 patients)

| | C [bootstrap- | | |
|---|---|---|---|
| | corrected | MR HR (95% CI); | |
| model | concordance index] | MR pval | AIC |
| spline(MR, k = 4) | 0.69 | 5.36 (1.78, 16.10); | 254.36 |
| * <MAF1> + cov | | .002 | |
| atan(MR) | 0.66 | 2.60 (1.24, 5.46); | 262.11 |
| * <MAF1> + cov | | .028 | |
| atan(MR) + cov | 0.64 | 2.71 (1.33, 5.49); | 260.48 |
| | | .006 | |
| MR (>50%) | 0.66 | 3.73 (1.46, 9.53); | 259.05 |
| * <MAF1> + cov | | .017 | |
| MR (>50%) + cov | 0.63 | 2.89 (1.29, 6.48); | 260.75 |
| | | .009 | |

As shown in table 4, TTD outcome metric prediction (51 events among 81 patients). Table shows that models that include baseline ctDNA level interaction with ctDNA change (MR in table) have superior performance in prediction/association with TTD per concordance index and AIC over models with only ctDNA change. Covariates (cov) included in the analysis include LOT, gender, age, and a weighted comorbidity index. MR (>50% decrease) denotes the categorical variable after thresholding Guardant Response MR score by greater than or equal and less than 50% decrease in ctDNA between timepoints.

TABLE 4
TTD outcome metric prediction (51 events among 81 patients)

| | C [bootstrap- | MR HR | |
|---|---|---|---|
| | corrected | (95% CI); | |
| model | concordance index] | MR pval | AIC |
| spline(MR, k = 4) | 0.66 | 2.37 (1.13, 4.98); | 361.29 |
| * <MAF1> + cov | | .014 | |
| atan(MR) | 0.62 | 1.96 (1.07, 3.60); | 363.40 |
| * <MAF1> + cov | | .057 | |
| atan(MR) + cov | 0.60 | 2.20 (1.22, 3.95); | 363.79 |
| | | .009 | |
| MR (>50% decrease) | 0.63 | 2.21 (1.12, 4.35); | 361.88 |
| * <MAF1> + cov | | .042 | |
| MR (>50% | 0.58 | 1.98 (1.07, 3.69); | 365.83 |
| decrease) + cov | | .003 | |

atan = arc tangent transformation function, MR = molecular response with MR Score = mean treatment VAF/mean baseline VAF, cov = covariates, CI = confidence interval, CPH = Cox proportional hazards model, ctDNA = circulating cell-free tumor DNA, G360 = Guardant360, HR = hazard ratio, ICI = immune checkpoint inhibitor, CRC = colorectal cancer, TTNT = time to next treatment, AIC = Akaike Information Criterion.

As shown in table 5, incorporation of both baseline ctDNA and ctDNA change metric improves prediction of patients that are at high risk of progression. High baseline ctDNA molecular nonresponders (N/H) have 14 times the hazard for TTNT event (proxy for progression) compared to low baseline ctDNA molecular responders (R/L) in the TKI cohort. Abbreviations: Molecular responders (R), nonresponders (N), low baseline ctDNA (L), high baseline ctDNA (H), responder/low baseline (R/L), non-responder/high baseline (N/H); NR=Not reached.

TABLE 5
Hazard of rwTTNT

| | | Reference | | CPH HR, | Median TTNT |
|---|---|---|---|---|---|
| Cohort | Group | Group | CPH HR | p-value | in months [CI] |
| ICI | N | R | 2.83 | <0.005 | N: 7.9 |
| | | | [1.58-5.09] | | [6.4-11.6]; |
| | | | | | R: 19.0 |
| | | | | | [11.2-NR]] |
| ICI | H | L | 1.76 | 0.056 | H: 9.1 |
| | | | [0.99-3.15] | | [7.5-19.9]; |
| | | | | | L: 19.0 |
| | | | | | [11.2-NR] |
| ICI | N/H | R/L | 4.31 | <0.005 | N/H: 5.7 |
| | | | [1.97-9.43] | | [4.4-8.7]; |
| | | | | | R/L: 19.0 |
| | | | | | [11.2-NR] |
| TKI | N | R | 4.28 | <0.005 | N: 4.0 |
| | | | [2.18-8.40] | | [3.4-5.8]; |
| | | | | | R: 14.6 |
| | | | | | [9.8-23.6] |
| TKI | H | L | 2.88 | 0.019 | H: 6.0 |
| | | | [1.19-6.98] | | [4.0-8.5]; |
| | | | | | L: NR |
| | | | | | [4.8-NR] |
| TKI | N/H | R/L | 14.86 | <0.005 | N/H: 4.0 |
| | | | [3.19-69.15] | | [3.2-5.8]; |
| | | | | | R/L: NR |
| | | | | | [5.8-NR] |

As shown in table 6, similar to continuous variable for ctDNA change in Table 1, CPH model with both MR and MVAF has trend towards higher concordance index and reduced AIC as compared to the same model but including only ctDNA change alone (MR) or baseline ctDNA level alone (maxVAF). In the model shown an interaction effect between ctDNA change (MR) and baseline ctDNA level was not included. However p-value for an interaction was borderline significant. Less clear interaction between the categorical form of the variable compared to the continuous form of the variables is expected. p-value in combined model was <0.005 for both cohorts. MVAF p-value in combined model was borderline significant (0.068, 0.063)

TABLE 6
CPH model

| Cohort | CPH Model | C-index | AIC | CPH HR |
|---|---|---|---|---|
| ICI | MR + MVAF + covariates | 0.65 | 418 | N = 2.82; |
| | | | | H = 1.72 |
| ICI | MR + covariates | 0.64 | 418 | N = 2.83 |
| ICI | MVAF + covariates | 0.58 | 427 | H = 1.76 |
| TKI | MR + MVAF + covariates | 0.76 | 366 | N = 3.99; |
| | | | | H = 2.36 |
| TKI | MR + covariates | 0.76 | 369 | N = 4.28 |
| TKI | MVAF + covariates | 0.63 | 381 | H = 2.88 |

1. A method, comprising:
receiving, by a computer system, genetic information of a subject comprising data taken at two or more time points and wherein cancer is detected in the subject;
extracting one or more features from the genetic information, the one or more features identified from a plurality of samples obtained from the subject;
generating, by a first classifier implemented by the computer system using a first machine learning algorithm, a first output indicating a first classification of the subject;
generating, by a second classifier implemented by the computer system using a second machine learning algorithm, a second output indicating a second classification of the subject;
identifying, by the computer system and from a population, additional subjects with genetic information that match the subject's genetic information based on the first classification and the second classification;
determining, by the computer system, at least one score with respect to the subject based additional subjects with matching genetic information;
determining, by the computer system, a composite score using the at least one score; and
determining, by a recommender implemented by the computer system, a recommendation indicating a treatment for the subject based on the composite score.

2. The method of claim 1, wherein the one or more features comprise a first mutant allele fraction (MAF) and a second MAF, each from the at two or more time points.

3. The method of claim 2, wherein the at least one score is based on a first mutant allele fraction (MAF) and a second MAF, a weighted mean of the first MAFs and a weighted mean of the second MAFs.

4. The method of claim 3, wherein the at least one score is based on a ratio of the weighted mean of the first MAFs and the weighted mean of the second MAFs and the confidence interval.

5. The method of claim 2, wherein the at least one score is based on a first mutant allele fraction (MAF) at the first time point and a second MAF at the second time point, a first central tendency measure of the first MAFs and a second central tendency measure of the second MAFs.

6. The method of claim 5, wherein the at least one score is based on a ratio of the first central tendency measure at the first time point to the second central tendency measure at the second time point.

7. The method of claim 6, wherein the central tendency measure is one or more of a: mean, median, or mode.

8. The method of claim 1, further comprising comparing a molecular response score for the subject having the cancer to a predetermined cutoff point to identify that the subject is a likely responder to one or more therapies for the cancer when the molecular response score is below the predetermined cutoff point or that the subject is a likely non-responder to the one or more therapies for the cancer when the molecular response score is at or above the predetermined cutoff point.

9. The method of claim 8, wherein the one or more therapies comprise one or more immunotherapies.

10. The method of claim 8, further comprising administering one or more therapies for the cancer to the subject in view of the at least one score.

11. The method of claim 10, further comprising discontinuing administering one or more therapies for the cancer to the subject in view of the at least one score.

12. The method of claim 1, comprising using the at least one score as a prognostic biomarker and/or a predictive biomarker for the subject.

13. The method of claim 4, comprising using a molecule count to calculate the standard deviation for a ratio in a set of MAF ratios.

14. The method of claim 4, comprising propagating a variance through a ratio in a set of MAF ratios.

15. The method of claim 1, further comprising excluding one or more germline and/or clonal hematopoietic variants when determining the mutant allele frequencies (MAFs).

16. The method of claim 2, wherein the first time point comprises a pre-treatment time point and wherein the second time point comprises an on- or post-treatment time point.

17. The method of claim 1, comprising generating the genetic information from nucleic acid molecules obtained from one or more tissues or cells in at least one sample.

18. The method of claim 17, comprising generating the genetic information from cell-free nucleic acids (cfNAs) in the at least one sample.

19. The method of claim 18, wherein the cfNAs comprise circulating tumor DNA (ctDNA).

20. The method of claim 1, wherein the first and/or second classifier is each implemented by a machine learning algorithm.

21. The method of claim 20, wherein the machine learning algorithm comprises a neural network, a support vector machine, a Hidden Markov Model, or a random forest model.

22. The method of claim 1, wherein the at least one score corresponds to a level of responsiveness to a treatment from a plurality of levels of responsiveness to the treatment.

23.-26. (canceled)

27. A computer readable medium comprising non-transitory computer executable instructions which, when executed by at least one electronic processor, perform the method of claim 1.

28. A system configured to perform the method of claim 1.
